(19) United States
(12) Patent Application Publication
(10) Pub. No.: US 2021/0361570 A1
(43) Pub. Date: Nov. 25, 2021

(54) IN SITU GELLING POLYSACCHARIDE-BASED NANOPARTICLE HYDROGEL COMPOSITIONS, AND METHODS OF USE THEREOF

(71) Applicant: McMaster University, Hamilton (CA)

(72) Inventors: Todd Hoare, Ancaster (CA); Ali Babar, Mississauga (CA); Michael J. Majcher, Hamilton (CA); Xiaoyun Li, Hamilton (CA); Andrew D. Lofts, Port Elgin (CA); Matthew A. Campea, Mississauga (CA)

(21) Appl. No.: 17/323,659

(22) Filed: May 18, 2021

Related U.S. Application Data
(60) Provisional application No. 63/027,112, filed on May 19, 2020.

(51) Int. Cl. A61K 9/06; A61K 47/36; A61K 9/51; B82Y 5/00

(52) U.S. Cl. CPC A61K 9/06; B82Y 5/00; A61K 9/5161; A61K 47/36

(57) ABSTRACT

The present application relates to in situ gelling hydrogel structures formed by the crosslinking of polysaccharide-based nanoparticles and functional polymers. Such systems can be designed to release the nanoparticles and/or encapsulated therapeutics either over time or in response to environmental stimuli.

### CROSS-REFERENCE TO RELATED APPLICATIONS

The present application claims the benefit of priority from co-pending U.S. provisional application No. 63/027,112 filed on May 19, 2020, the contents of which are incorporated herein by reference in their entirety.

### FIELD

The present disclosure relates to hydrogels formed by mixing at least one polysaccharide-based nanoparticle and at least one secondary polymer precursor that can form an in situ-gelling hydrogel with utility in drug delivery and other biomedical applications. In particular, the present disclosure is directed to a hydrogel composition comprising a polysaccharide-based nanoparticle functionalized with a first functional moiety and a polymer functionalized with a second functional moiety, wherein the first functional moiety and the second functional moiety are crosslinked through reversible covalent and/or physical crosslinks to form the hydrogel composition.

### BACKGROUND

Hydrogels are porous, hydrophilic, crosslinked 3D polymer networks.¹ Crosslinking can occur through a number of mechanisms, for example electrostatic interactions, covalent bonds, supramolecular chemistry, hydrogen bonding, pi-pi stacking, and more. The tunable mechanical properties of hydrogels make them promising for use in a wide range of biological applications such as tissue engineering,² biosensors,³ and drug delivery.⁴ For example, in drug delivery, hydrogel swelling and porosity allows for facile loading of hydrophilic drugs, while stimuli-responsive crosslinks allow for the selective formation and degradation of hydrogels within the site of tissue target. Hydrogels can be made as a bulk phase or on the micro/nanoscale, all of which offer advantages in particular applications.¹ For example, in drug delivery, nanoscale hydrogels (nanogels) offer improved circulation prior to tissue targeting, while bulk gels can be implanted and offer sustained drug release in a single target site.

A nanocomposite hydrogel system is a system in which one of the components of the hydrogel is a nanoparticle. A nanoparticle network hydrogel is a particular type of nanocomposite hydrogel system in which a network is formed from the crosslinking of at least one nanoscale building block.⁵ The major distinction of these nanoparticle network hydrogels from other nanocomposite hydrogels is that in the former the nanoparticles are crosslinked together (either directly or by bridging them with one or more other polymer(s)) to preserve the inherent nanoparticle length scale while also introducing higher-order nanoparticle structuring while in the latter (a more general term) nanoparticles may be incorporated either via crosslinking or via physical encapsulation. This direct crosslinked structure leads to unique properties such as higher conductivity or charge⁶, increases in mechanical strength⁷, more tunable porosity for drug delivery/release^{8,9}, improved filtration of aqueous organics/pollutants¹⁰, reduced bio-fouling¹¹, and/or unique swelling properties in different media¹², depending on the materials used and the targeted application. Given these properties, nanoparticle network hydrogels show potential in biomedical, industrial, catalytic, environmental, agricultural, and other applications.

Fabricating hydrogels using nanoparticle building blocks also offers significant benefits in the context of drug delivery. For example, nanoparticles (NPs) have been widely demonstrated to be useful drug delivery vehicles due to their ability to freely circulate through blood vessels and, ultimately, through tight cellular junctions. However, a meta-analysis done over the last decade for anti-cancer nanoparticle therapeutics showed that only 0.7% of nanoparticle-based drug delivery vehicles reaches the tumor target.¹³ A primary hypothesis to account for this inefficiency is the different nanoparticle size requirements for long-term circulation and delivery to targeted sites in the body. For example, a nanoparticle is most effective at evading macrophage-associated phagocytosis when it is <200 nm in diameter, avoiding entering off-target tissues >100 nm in diameter, and entering hard-to-reach cells of tumour tissues when <50 nm in diameter.¹⁴⁻¹⁷ Furthermore, sub-50 nm NPs possess a lower tendency to extravasate from blood vessels compared with large-size nanoparticles (100-200 nm), which could improve the accumulation of NPs in tumor tissues. Although small nanoparticles have shown capacity for deep penetration into tumor tissues, they could be rapidly cleared by the reticuloendothelial system in vivo to result in insufficient accumulation at the tumor site.¹⁸⁻²⁰ Together, these data suggest that a size-switching nanoparticle could improve the efficacy of drug delivery vehicles.

In addition to size-switching, NP surface chemistry properties must be considered for accurately targeting and entering tumor tissues. For example, binding (and subsequent endocytosis) in tumours but not off-target tissues can occur by binding to ligands that are upregulated in tumour environments. For example, cluster of differentiation 44 (CD44) is a tumour-upregulated cell surface receptor that actively transports glycosaminoglycans into the tumour microenvironment.²¹ Similar endocytotic effects occur with glucose-based polysaccharides through the glucose transporter (GLUT) protein family²² or positively charged particles bound to negatively charged oncocyte exteriors.²³ Once endocytosed, the tumour (or similar pH) microenvironment can selectively de-crosslink the nanoparticle/nanogel to increase the diffusion of drug into the tumour site. Materials crosslinked with disulfide bonds can be degraded by glutathione (GSH) and the cysteine/cystine (Cys/CySS) tumour-upregulated peptides that rapidly reduce disulfide bonds in vivo;²⁴⁻²⁷ alternately, the acidic tumour environment rapidly reduces a wide range of pH-responsive bonds.²⁸ Reducing disulfide bonds intracellularly (via GSH) allows for drug release directly into the cell, while reducing extracellularly (via Cys/CySS) may allow for released payloads to reach other areas of the tumour mass, especially if payload is below 50 nm in size. Alternately, in other contexts, slow degradation of highly labile bonds (e.g. Schiff bases) can meter release of nanoparticles over time from a depot that enables continual nanoparticle release at a local site. In any of these contexts, designing materials that are initially either bulk gels or micro/nanoparticles with the potential to either localize or circulate but can be degraded over time or in that microenvironment to release small nanoparticles that can improve penetration, improve cell uptake, and/or alter delivery kinetics offers potential in improving the efficacy of drug therapies.

Polysaccharides, chemically defined as a polyhydryaldehyde or polyhydroxyketone²⁹⁻³⁰ are widely used materials in biomedical applications due to their generally high cytocompatibility and degradability via oxidative, enzymatic, or hydrolytic processes in the body. Starch is among the most abundant storage polysaccharides³¹ consisting of anhydrous glucose units joined together by alpha-D-(1->4) and alpha-D-(1->6) glycosidic ether linkages³². The ratio of linear alpha-D-(1->4) linkages (amylose) to branched alpha-D-(1->6) linkages (amylopectin)³² is unique to the source of starch, yielding different crystallinities and physical properties.³⁴ Starch shows particular promise as a biomaterial as it is enzymatically degradable, non-cytotoxic,³⁵ and has low inflammation and protein adsorption.³⁶ However, some properties of unmodified starch provide inherent challenges to its use as a drug delivery vehicle, such as its high viscosity and (semi-)crystalline structure that limit its processability and functionalizability; such viscosity challenges are also observed in using other types of polysaccharides, in particular high molecular weight and highly water-binding polysaccharides such as hyaluronic acid. The use of nanoparticles/nanogels can overcome these challenges. Polysaccharide nanoparticles remain minimally viscous even at high concentrations (allowing facile processability on an industrial scale) while also remaining highly hydrated, providing them with gel-like compressibility that enables (for example) penetration through tight cellular junctions and ultimate transport to hard-to-reach areas of target tissues³⁷⁻³⁸. In addition, specific to polysaccharides like starch that crystallize and are thus typically hard to functionalize, nanoparticles can be processed in a manner such that they are amorphous to enable functionalization using simple chemical methods.

### SUMMARY

The present disclosure relates to in situ gelling hydrogel compositions comprising at least one functionalized polysaccharide-based nanoparticle and at least one complementary polymer precursor, in which functionalization of one or both of the polysaccharide-based nanoparticle or complementary polymer precursor enables the formation of a crosslinked network. Such crosslinks may be formed via covalent or non-covalent bonds. In some embodiments, reversible covalent crosslinks including functional group crosslinking and/or in situ gelling (“click”) crosslinking via Michael addition, disulfides, imines, hydrazones, oximes, thioacetals, [2+4] Diels-Alder cycloaddition, and alkyne-azide chemistry. In other embodiments, physical crosslinks such as ionic/electrostatic interactions, stereocomplexation, hydrogen bonding, host-guest interactions, hydrophobic interactions, pi-pi stacking, and metal-ligand coordination are used. In an embodiment, the polysaccharide-based nanoparticle is primarily comprised of starch.

In one embodiment, the crosslinking of the hydrogel precursors allows for the entrapment of drugs or therapeutics. In another embodiment, the functionalization of the precursor materials or the physical entrapment itself may protect the loaded drugs from chemical denaturation or slow their release from the hydrogel. In another embodiment, the functionalization allows for tunable environment-based responses, including but not limited to rapid degradation, when exposed to specific biologically relevant conditions, including but not limited to specific pH values, enzyme concentrations, or molecule concentrations.

In one aspect of the disclosure, the hydrogel is formed using in situ-gelling pairs of functionalized precursor polymers and/or functionalized polysaccharide-based nanoparticles that can spontaneously crosslink upon co-delivery or sequential delivery to the target site to form a hydrogel. In one embodiment, co-delivery is achieved using a double-barrel syringe. In another embodiment, the gel can be formed via sequential delivery of the precursor polymers via pipetting, spraying, injection, or any other suitable delivery mechanism.

In a further aspect of the disclosure, the therapeutic may be chosen among drugs, proteins, antibodies, enzymes, peptides, DNA, RNA, aptamers, other polynucleotides, carbohydrates, glycoproteins, proteoglycans, or any other molecule with relevant bioactivity (i.e. enzymatic, binding affinity, transport, etc.) useful in a specific application. In some embodiments, the therapeutic is chosen to target the central nervous system (CNS). In other embodiments, the therapeutic is chosen to treat cancer. The therapeutic may be physically mixed with one or more of the precursor polymers and/or sequentially added to the pre-polymers prior to hydrogel formation to enable physical immobilization within the gel network. In one embodiment, functional groups native to or incorporated within one or more of the precursor polymer(s) may be used to promote physical interactions with the therapeutic and thus alter its retention and/or release characteristics and/or stability inside the network. In another embodiment, the biomolecule can be covalently tethered to the gel network either prior to or during network formation.

In one embodiment of the present disclosure, the hydrogel is a bulk hydrogel fabricated by mixing one or more functionalized polysaccharide-based nanoparticle(s) with a complementary linear polymer. In one embodiment, this hydrogel composition is comprised of
a. a polysaccharide-based nanoparticle that is aldehyde-functionalized, and
b. at least one polymer containing amine moieties, including but not limited to carboxymethyl chitosan and chitosan, wherein
c. the polysaccharide-based nanoparticle and second polymer are crosslinked through imine bonds.

In another embodiment, one or more of the precursor materials is additionally functionalized with a group to prevent chemical denaturation or oxidation of loaded drugs or loaded with an excipient compound that stabilizes loaded drugs. In an embodiment, the polysaccharide-based nanoparticle is functionalized with a boronic acid moiety.

In one embodiment, the present disclosure is used as an in situ gelling nasal spray to treat a central nervous system condition in which
a. the condition can be treated by administering an antipsychotic drug to the brain.
b. the condition can be treated by administering a dopamine agonist to the brain.

In another embodiment, the hydrogel composition is comprised of
a. at least one precursor polymer that is a thiol functionalized glycosaminoglycan,
b. a polysaccharide-based nanoparticle that may or may not be functionalized with thiol groups, wherein
c. the hydrogel is crosslinked via disulfide bonds

In another embodiment, the hydrogel composition is a mixture comprising
a. at least one cationic polysaccharide-based nanoparticle or precursor polymer
b. at least one anionic polysaccharide-based nanoparticle or precursor polymer, wherein
c. the cationic and anionic precursor materials are physically crosslinked through electrostatic interactions.

In another embodiment, the anionic functional group is pH-responsive in a pH range relevant to physiological pH gradients in infection sites, cancer tumors, and/or other disease sites.

In such an embodiment as described above, the hydrogel is used as an anti-cancer therapeutic that can be degraded in an accelerated manner in the microenvironment of a tumor.

In another aspect of the disclosure, gelation is performed within a water-in-oil emulsion that can template the formation of a gel particle that may have any dimension provided that it is larger than the constituent polysaccharide-based nanoparticles. In an embodiment, the gel particle is a microgel or nanogel. The emulsion can be formed using any method known in the art, including but not limited to sonication, homogenization, microfluidization, or other forms of mixing, to form an emulsion, microemulsion, miniemulsion, or nanoemulsion. In an embodiment, sonication is used to create a miniemulsion between water and an organic oil.

In an embodiment, the gel particle is a nanocluster fabricated with a diameter less than 1000 nm, or less than 250 nm, or less than 150 nm, or less than 100 nm.

In an embodiment, a nanocluster with a diameter of 150-200 nm is fabricated from nanoparticles with diameters of 20-50 nm.

In one embodiment, the microgel or nanogel composition is comprised of
a. a polysaccharide-based nanoparticle that is aldehyde-functionalized, and
b. at least one polymer containing amine moieties, including but not limited to carboxymethyl chitosan or chitosan, wherein
c. the polysaccharide-based nanoparticle and second polymer are crosslinked through imine bonds.

In an embodiment, the sizes of the nanocluster and polysaccharide-based nanoparticle are selected such that the sizes will induce different biodistributions when applied to the body.

In an embodiment, the nanocluster size is selected for long-term circulation (about 10-500, or about 50-250 nm) while the polysaccharide-based nanoparticle size is selected for high tissue penetration (less than 100 nm, less than 50 nm, or less than 25 nm).

In an embodiment, the microgel or nanogel composition is used as a therapeutic to treat central nervous system disorders.

In another embodiment, the microgel or nanogel composition is comprised of
a. a precursor polymer that is a thiol functionalized glycosaminoglycan,
b. a polysaccharide-based nanoparticle that may or may not be functionalized with thiol groups, wherein
c. the nanogel is crosslinked via disulfide bonds

In an embodiment, the disulfide-crosslinked microgels or nanogels are prepared with the aid of a gelator precursor used to catalyze disulfide formation between thiol-functionalized glycosaminoglycans.

In another embodiment, the microgel or nanogel composition is a mixture comprising
a. at least one cationic polysaccharide-based nanoparticle or precursor polymer
b. at least one anionic polysaccharide-based nanoparticle or precursor polymer, wherein
c. the cationic and anionic precursor materials are crosslinked through electrostatic interactions.

In another embodiment, the anionic functional group is pH-responsive in a pH range relevant to physiological pH gradients in infection sites, cancer tumors, and/or other disease sites.

In such an embodiment as described above, the microgel or nanogel is used as an anti-cancer therapeutic that can be degraded in an accelerated manner in the microenvironment of a tumor.

In an embodiment, the present disclosure also includes a double-barreled syringe delivery method, comprising
a. at least one functionalized precursor particle or polysaccharide-based nanoparticle in one barrel,
b. at least one functionalized precursor polymer or polysaccharide-based nanoparticle with complementary functionality to form a hydrogel in the second barrel, wherein
c. upon injection through a needle, cannula, catheter, tube, nebulizer, or other delivery device, the first and second precursor solution form a hydrogel.

In another embodiment, the present disclosure includes the use of an inverse miniemulsion to template the formation of microgels or nanogels comprising
a. an oil phase constituting the majority of the volume,
b. an aqueous phase comprised of the at least one polysaccharide-based nanoparticle and at least one crosslinkable precursor polymer, and
c. at least one emulsifier to stabilize the aqueous droplets formed upon shearing, wherein
d. the size of the microgel or nanogel is controlled based on the energy used for mixing.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows the gelation of aldehyde-modified starch nanoparticles and O-carboxymethyl chitosan in an embodiment of the disclosure.

FIG. 2 shows the synthesis of aldehyde-functionalized starch nanoparticles and phenylboronic acid-functionalized starch nanoparticles and subsequent crosslinking with carboxymethyl chitosan via Schiff bases to form a hydrogel that promotes interaction with drugs containing vicinal diols to stabilize the drug in an embodiment of the disclosure.

FIG. 3 shows a representative transmission electron microscopy (TEM) image of aldehyde-functionalized starch nanoparticles in the presence of background latex nanoparticles in an embodiment of the disclosure.

FIG. 4 shows the gelation kinetics of various aldehyde-functionalized starch nanoparticles with various concentrations of carboxymethyl chitosan in an embodiment of the disclosure.

FIG. 5 shows the gelation kinetics of various aldehyde-functionalized (top) and phenylboronic acid-functionalized (bottom) starch nanoparticles with various concentrations of carboxymethyl chitosan in embodiments of the disclosure.

FIG. 6 shows the gelation kinetics of various aldehyde-functionalized starch nanoparticles with various concentrations of carboxymethyl chitosan (CMCh) variant carboxymethyl chitosan oligosaccharide lactate (COL) in an embodiment of the disclosure.

FIG. 7 shows the degradation kinetics of hydrogels prepared with various types and concentrations of aldehyde-functionalized starch nanoparticles crosslinked various concentrations of carboxymethyl chitosan containing POAPA in an embodiment of the disclosure.

FIG. 8 shows the degradation kinetics of hydrogels prepared with various types and concentrations of aldehyde-functionalized starch nanoparticles crosslinked various concentrations of carboxymethyl chitosan containing POAPA in an embodiment of the disclosure.

FIG. 9 shows the storage modulus of hydrogels prepared from aldehyde-functionalized starch nanoparticles at various concentrations and degrees of functionalization with carboxymethyl cellulose in an embodiment of the disclosure.

FIG. 10 shows force versus distance curves related to pulling aldehyde-functionalized starch nanoparticle/carboxymethyl chitosan hydrogels prepared with different carboxymethyl chitosan concentrations from a mucin-coated poly(dimethylsiloxane) surface in an embodiment of the disclosure.

FIG. 11 shows the relationship between spray area and spray distance for aldehyde-functionalized starch nanoparticle/carboxymethyl chitosan hydrogels delivered via an aerosolizing nozzle attached to a standard syringe in an embodiment of the disclosure.

FIG. 12 shows the degradation of hydrogels prepared from aldehyde-functionalized starch nanoparticles and carboxymethyl chitosan using dynamic light scattering in an embodiment of the disclosure.

FIG. 13 shows the degradation of hydrogels prepared from aldehyde-functionalized starch nanoparticles and carboxymethyl chitosan using transmission electron microscopy in an embodiment of the disclosure.

FIG. 14 shows the in vitro cytotoxicity of the aldehyde-functionalized starch nanoparticles to SH-SY5Y human neuroblastoma precursor cells (upper) and hNEpC human nasal epithelial cells (lower) in an embodiment of the disclosure.

FIG. 15 shows the degradation and normalized equilibrium absorbance of PAOPA loaded hydrogels prepared from aldehyde-functionalized starch nanoparticles and carboxymethyl chitosan measured with HPLC in an embodiment of the disclosure.

FIG. 16 is a schematic showing the mechanism of both hydrogel formation and its subsequent route of administration for intranasal delivery in an embodiment of the disclosure.

FIG. 17 shows a graphical overview of the experimental procedure for the use of aldehyde-functionalized starch nanoparticles crosslinked with carboxymethyl chitosan for intranasal delivery of antipsychotic drugs in an embodiment of the disclosure.

FIG. 18 shows social interaction behavioral testing data for all of the drug, precursor polymer, and hydrogel formulations based on aldehyde-functionalized starch nanoparticles and carboxymethyl chitosan for intranasal delivery of the anti-schizophrenic drug (3R)-2-oxo-3-[[(2S)-2-pyrrolidinylcarbonyl]amino]-1-pyrrolidineacetamide (PAOPA) in an embodiment of the disclosure.

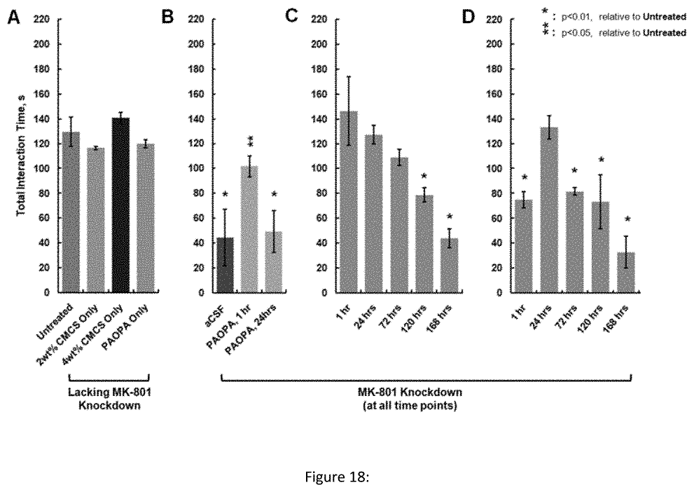

FIG. 19 shows the biodistribution of fluorescently labeled aldehyde-functionalized starch nanoparticles after intranasal administration of an aldehyde-functionalized starch nanoparticle/carboxymethyl chitosan hydrogel in an embodiment of the disclosure.

FIG. 20 shows the potential of hydrogels based on aldehyde-functionalized starch nanoparticles and carboxymethyl chitosan to reduce the oxidation of levodopa, with or without nanoparticle functionalization with phenylboronic acid in an embodiment of the disclosure.

FIG. 21 shows the results of a C. elegans in vivo test confirming the improved stability of levodopa in hydrogels based on aldehyde-functionalized starch nanoparticles and carboxymethyl chitosan in an embodiment of the disclosure.

FIG. 22 shows the preparation methods for fabricating thiolated chondroitin sulfate in an embodiment of the disclosure.

FIG. 23 shows a schematic of the inverse miniemulsion technique for fabricating nanocluster nanogels based on starch nanoparticles and thiol-functionalized chondroitin sulfate in an embodiment of the disclosure.

FIG. 24 shows the particle sizes of nanocluster nanogels based on starch nanoparticles and thiol-functionalized chondroitin sulfate in an embodiment of the disclosure.

FIG. 25 shows the drug loading capacity of doxorubicin hydrochloride into chondroitin sulfate-based nanocluster nanogels in an embodiment of the disclosure.

FIG. 26 shows the drug release capacity of doxorubicin hydrochloride from starch nanoparticle/thiol-functionalized nanocluster nanogels at various concentrations of glutathione in an embodiment of the disclosure.

FIG. 27 shows the cell viability of doxorubicin-loaded, chondroitin sulfate-based nanogels in NIH 3T3 fibroblast, B16-F10 melanoma, and CT26 colon carcinoma cell lines in an embodiment of the disclosure.

FIG. 28 is a schematic representation illustrating one example of the synthesis of pH-responsive poly(oligoethylene glycol) polymers with reversible or irreversible charge between normal physiological and mildly acidic pH in an embodiment of the disclosure.

FIG. 29 is a schematic representation of the synthesis of aldehyde-functionalized cationic starch nanoparticles in an embodiment of the disclosure.

FIG. 30 shows an example of loading the anti-cancer drug doxorubicin into an aldehyde-functionalized cationic starch nanoparticle in an embodiment of the disclosure.

FIG. 31 shows an example of forming a doxorubicin-loaded smart nanocluster nanogels through electrostatic interactions between cationic starch nanoparticles and anionic poly(oligoethylene glycol methacrylate) polymers in an embodiment of the disclosure.

FIG. 32 shows the size distributions of nanocluster nanogels prepared from cationic starch nanoparticles and anionic poly(oligoethylene glycol methacrylate) in an embodiment of the disclosure.

FIG. 33 shows the cell viability of B16-F10 mouse skin melanoma cells at different incubation times with free doxorubicin compared to nanocluster nanogels prepared from cationic starch nanoparticles and anionic poly(oligoethylene glycol methacrylate) in an embodiment of the disclosure.

FIG. 34 shows the penetration of doxorubicin-loaded nanocluster nanogels prepared through electrostatic interactions between cationic starch nanoparticles and anionic poly(oligoethylene glycol methacrylate) polymers with reversible or irreversible charge between normal and mildly acidic pH into B16-F10 cell spheroids at physiological relative to tumoral pH in an embodiment of the disclosure.

FIG. 35 shows the anti-tumor effects of the doxorubicin alone relative to doxorubicin-loaded nanocluster nanogels prepared through electrostatic interactions between cationic starch nanoparticles and anionic poly(oligoethylene glycol methacrylate) polymers with reversible or irreversible charges in an embodiment of the disclosure.

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “derivative” as used herein refers to a substance which comprises the same basic carbon skeleton and functionality as the parent compound but can also bear one or more substituents or substitutions of the parent compound.

The term “w/w” as used herein means the number of grams of solute in 100 g of solution.

The term “w/v” as used herein refers to the number of grams of solution in 100 mL of solvent.

The term “polysaccharide” as used herein refers to any polymer of, for example, at least 10 repeat units in which the repeat unit(s) consist of one or more type(s) of carbohydrate.

The term “nanoparticle” as used herein refers to any material with at least one dimension (such as the diameter, length, width, depth) less than one micron.

The term “polysaccharide-based nanoparticle” as used herein refers to any nanoparticle in which the majority, or all, of its mass is comprised of one or more polysaccharide components.

The term “starch nanoparticle” (SNP) as used herein refers to a polysaccharide nanoparticle based on starch from any source with any degree of amylopectin and amylose content.

The term “hydrogel” as used herein refers to a crosslinked network that imbibe water. The dimensions of the hydrogel vary from the bulk scale to the nanoscale.

The term “bulk gel” as used herein refers to a crosslinked hydrogel without one dimension (such as the diameter, length, width, depth) on the nanometer scale.

The term “microgel” or “nanogel” as used herein refers to a crosslinked hydrogel with one dimension (such as the diameter, length, width, depth) on the micrometer or nanometer scale respectively.

The term “nanocluster” as used herein refers to a group of any number of nanoparticles and/or nanogels physically or chemically associated to comprise a larger nanoparticle.

The term “crosslinked” or “crosslink” as used herein is defined as a bond and/or attraction that links a first functional moiety to a second functional moiety. The bonds can be covalent or non-covalent bonds.

The term “de-crosslinked” or “de-crosslinking” as used herein refers to the breaking of a crosslink (for example, the inverse of “crosslinked” or “crosslinking” or “re-crosslinking”), yielding the deformation of the original hydrogel or nanogel structure. For example, in one embodiment, disulfide crosslinked polymer is “de-crosslinked” in the presence of GSH, a disulfide reducing peptide.

The term “gelator” as used herein refers to a chemical compound that accelerates the rate of crosslinking in a hydrogel (i.e. from hours to minutes).

The term “thiolated” or “thiol-functionalized” as used herein refers to a compound that has been chemically modified to incorporate a thiol group into its structure. Thiolation (i.e. the process of thiol functionalizing a compound) can occur through reactions such as (but not limited to) reductive amination, carbodiimide conjugation, disulfide reduction, sulfate reduction and the like.

The term “disulfide” as used herein refers to a reactive group where two thiol groups have bonded together, yielding a covalent sulfur-sulfur bond.

The term “inverse miniemulsion” or “water-in-oil emulsion” as used herein refers to the process by which water and water-soluble chemicals are stably dispersed in an oil-based medium, including emulsifiers to stabilize the interface. Adding energy to this dispersion alters the size of the individual water droplets in the system. Energy can be added to this suspension through techniques such as homogenization, sonication, microfluidization, mechanical stirring, and the like.

The term “surfactant” or “emulsifier” as used herein refers to a compound of mixed composition that kinetically stabilizes the dispersion in an emulsion. The mixed composition refers to one end of the compound being hydrophilic while the other is hydrophobic.

The term “physically entrapped” as used herein refers to a nanoparticle being encapsulated by another material (such as a hydrogel) without forming a covalent or specific non-covalent bond with this compound. For example, in one embodiment, unfunctionalized starch nanoparticles are physically entrapped in a disulfide-crosslinked chondroitin sulfate matrix in which the thiolated chondroitin sulfate only forms disulfide bonds with itself and not with the starch nanoparticles.

The term “glycosaminoglycan” as used herein refers to a polysaccharide consisting of repeating disaccharide units with an amino sugar and either a galactose- or uronic acid-based sugar.

The term “electrophile-functionalized” or “electrophilic” as used herein refers to the chemical functionalization of a compound resulting in one or more of their chemical moieties to become electrophiles.

The term “nucleophile-functionalized” or “nucleophilic” as used herein refers to the chemical functionalization of a compound resulting in one or more of their chemical moieties to become nucleophiles.

The term “redox responsiveness” as used herein refers to the ability for a material to change its properties when in the presence of a compound that can either reduce or oxidize other compounds. This reduction or oxidation may lead to the formation and/or deformation of new chemical bonds in the process. Material properties that change may include (but are not limited to) size, zeta potential, electrophoretic mobility, viscosity, and mechanics.

The term “degree of substitution” as used herein refers to the average number of chemical groups functionalized per base unit of a given polymer. For example, a DS of 0.1 means that a particular functional group is introduced to a polymer on 1 of every 10 base units on average.

The term “milli-Q water (MQW)” or “deionized water (DIW)” as used herein refers to water that has been purified to remove ions and having a final resistivity of at least 18.2 MΩ/cm.

The term “size/surface switching” as used herein refers to the size and surface switching property of the nanoclusters. For example, in one embodiment, the size of the nanocluster nanogel switches from 128 nm to 14 nm and the surface charge of the nanocluster nanogel switches from negatively charged to positively charged.

The term “smart” as used herein refers to a polymeric nanoparticle that exhibits the ability to switch one or more physical properties, including but not limited to size, surface charge, refractive index, or chemistry, upon the application of a stimulus, including but not limited to pH, temperature, ionic strength, or the concentration of a chemical.

The term “responsive” as used herein refers to the chemical structure of hydrogel or one of its precursors that can respond to its local environment. For example, in one embodiment, the local environmental factor is pH, by which for example the poly[oligo(ethylene glycol) methyl ether methacrylate]-hydrazine-dimethylmaleic acid copolymer hydrolyzes and the to detach the dimethylmaleic acid group when the pH is changed from 7.4 to 6.5.

The term “copolymer” as used herein is defined as a polymer derived from two or more different monomers. For example, in one embodiment, a copolymer of the present disclosure includes a co-polymer of poly(ethylene glycol) methyl ether methacrylate (OEGMA) and acrylic acid.

The term “positively charged” as used herein refers to a nanoparticle or co-polymer precursor that has a net positive charge on its surface or within its polymer chain. For example, in one embodiment, the cationic starch nanoparticles used to fabricate electrostatically-crosslinked nanocluster nanogels have a net positive charge of about +5 to about +40 mV.

The term “negatively charged” as used herein refers to a nanoparticle or co-polymer precursor that has a net negative charge on its surface or within its polymer chain. For example, in one embodiment, the carboxyl groups of poly[oligo(ethylene glycol) methyl ether methacrylate]-hydrazine-dimethylmaleic acid copolymer (POEGMA-Hyd-DMA) has a net negative charge at pH 7.4.

The term “spheroid” as used herein refers to a three-dimensional tumor cell model that simulates a live cell's environment and 3D positioning in native tissues.

### Description of Materials and Synthesis of the Disclosure

The present disclosure is generally directed to a hydrogel composition comprising one or more primary precursor polysaccharide-based nanoparticle(s) (the polysaccharide-based nanoparticle) and polymer(s) or copolymer(s) (the secondary precursor material), wherein the primary and secondary precursor materials are crosslinked by one or more types of covalent and/or physical crosslinks. In an embodiment, the polysaccharide is starch.

In an embodiment, the crosslinks that form are degradable or reversible. In a further embodiment, the crosslinks can degrade or reverse under physiologically-relevant conditions to release the polysaccharide-based nanoparticles over time or upon the application of a biological stimulus.

In another embodiment, the precursor materials (the polysaccharide-based nanoparticle and the polymer) are combined in situ during administration of the product to form a bulk gel. Methods of such administration include but are not limited to intravenous, intramuscular, intracranial, subcutaneous, intradermal injection or intranasal spray using an aerosolization device. In a further embodiment, the precursors are stored in separate barrels of a double barrel syringe until they are mixed during the process of an injection via a static mixing device.

In another embodiment, the precursor materials (the polysaccharide-based nanoparticle and the polymer) are combined in situ during a process to form a nanocluster microgel or nanogel and administered after the crosslinks have formed.

In another embodiment, the resulting hydrogel can physically or chemically encapsulate therapeutic molecules or biomolecules for the treatment of a condition.

In one embodiment, the polysaccharide-based nanoparticle can covalently or non-covalently bond directly or indirectly to drugs/molecules. Non-covalent bonds include but are not limited to electrostatic attraction, van der Waals forces, hydrogen bonding, hydrophobic interactions, or host-guest interactions.

In one embodiment, the drug is encapsulated inside the hydrogel as to increase bioavailability or increase its stability. An example includes, but is not limited to, preventing drug oxidation via reversible covalent or non-covalent bonds with the drug or physical exclusion of the oxidative species.

In one embodiment, the drug loading efficiency can be controlled by the degree of functionalization and the concentration of the functionalized precursor polysaccharide-based nanoparticles. In one embodiment, the mass-based drug loading efficiency is about 0.5%, to about 5.0%, about 5.0% to about 10.0%, or about 10.0% to about 20.0%.

In one embodiment, the drug(s) loaded are drugs used to treat neurological disorders such as antipsychotics, mood stabilizers, and/or antidepressants, including but not limited to (3R)-2-oxo-3-[[(2S)-2-pyrrolidinylcarbonyl]amino]-1-pyrrolidineacetamide) (PAOPA), prolyl-leucyl-glycinamide, haloperidol, loxapine, chloropromazine, perphenazine, clozapine, olanzapine, risperidone, lurasidone, ziprasidone, and lithium. In a further embodiment, hydrogels containing one or more of these drugs are used to treat schizophrenia or bipolar disorder.

In one embodiment, the drug(s) loaded are dopamine agonists including but not limited to levodopa, carbidopa and benserazide. In a further embodiment, hydrogels containing one or more of these drugs are used to treat Parkinson's disease.

In one embodiment, the drug(s) loaded are chemotherapeutics. This includes but is not limited to doxorubicin, camptothecin, paclitaxel, sunitinib, or cisplatin. In a further embodiment, hydrogels containing one or more of these drugs are used to treat cancer.

In another embodiment, the functionalized polysaccharide-based nanoparticle and the polymer or copolymer (the polymer) represent both the hydrogel precursor polymers as well as the hydrogel degradation products.

In one embodiment, the polymer or copolymer (the polymer) has a molecular weight which is less than the molecular weight cut-off for renal (kidney) clearance. In another embodiment, the copolymer has a molecular weight which is less than about 60 kDa. In another embodiment, the copolymer has a molecular weight of about 30 kDa to about 60 kDa. In another embodiment, the copolymer has a molecular weight of about 5 kDa to about 30 kDa. In one embodiment, the degradation time of the hydrogel can be altered to control the rate at which a therapeutic is released. In one embodiment, the hydrogel composition is responsive to the surrounding environment. Examples of environmental triggers include but are not limited to local temperature, pH, ionic strength, or the concentration of an enzyme, receptor, molecule, biological, drug, or salt. In one such embodiment, the hydrogel degrades upon the addition of a reducing agent such as glutathione and can deliver more drug at a site of infection at which the body has increased glutathione concentrations. In another embodiment, hydrogels show accelerated degradation in aqueous solutions containing high concentrations of disulfide-reducing enzymes including but not limited to thioredoxin and glutaredoxin.

In one embodiment, the dimensions of both the hydrogel as well as the polysaccharide-based nanoparticles allow for biological advantage, such as (but not limited to) improved tissue/intratumor penetration, longer circulation times, or tunable mucoadhesion and/or mucopenetration. For example, in one embodiment, the size of a nanocluster nanogel is designed to meet the necessary requirements for long term circulation (typically about 50 nm to about 250 nm or about 100 nm to about 200 nm). In other embodiments, the polysaccharide-based nanoparticles are of appropriate size for high intratumoral or transmembrane permeation, typically about 10 nm to 60 nm or about 20 nm to about 40 nm.

In one embodiment the total concentration of polysaccharide-based nanoparticles in the hydrogel is between about 1 to about 60 w/v %, or about 2 to about 50 w/v %, or about 5 to about 30 w/v %.

In one embodiment, polysaccharide-based nanoparticles are functionalized with functional groups to enable crosslinking and/or alter the properties of the polysaccharide-based nanoparticle, including but not limited to aldehydes, bromobenzaldehydes, or derivative thereof, carboxylic acid groups, amino groups, phenylboronic acid groups, phosphate groups, sulfate groups, zwitterionic groups, or hydrophobic moieties.

In one embodiment, the degree of functionalization of the polysaccharide-based nanoparticle with a functional entity is between about 1% to about 50% of the total number of glucose repeat units, or between about 2% to about 30% of the total number of glucose repeat units, or between about 5% to about 20% of the total number of glucose repeat units.

In one embodiment, the polysaccharide-based nanoparticles are functionalized with at least two or at least three different functional groups, including but not limited to functionalizing different locations of the polysaccharide monomer unit(s) or having multiple functionalizations at the same monomer location.

In one embodiment, polysaccharide-based nanoparticles of two or more types or degrees of functionalization are used to prepare a single hydrogel, including but not limited to combining different concentrations of different polysaccharide-based nanoparticle suspensions varying in their degree of functionalization and/or functionalization moiety and/or drug loading.

In one embodiment, the polysaccharide-based nanoparticle precursor comprises a cationic polysaccharide-based nanoparticle with a zeta potential in the range of about +5 mV to about +50 mV or a anionic polysaccharide-based nanoparticle with zeta potential in the range of about −5 mV to about −50 mV. In a further embodiment the polysaccharide-based nanoparticle precursor could be additionally functionalized with groups to enhance drug loading, delivery kinetics, or stability, including but not limited to aldehyde-functionalized cationic polysaccharide-based nanoparticles to improve binding to amine-containing drugs.

In one embodiment, the polysaccharide-based nanoparticle is oxidized, for example using sodium periodate, to create molar equivalents of aldehyde groups ranging from about 0.25 to about 2-fold the number of carbohydrate repeat units in the polysaccharide.

In one embodiment, the polysaccharide-based nanoparticle is functionalized with carboxylic acid groups using chloroacetic acid with molar equivalents of about 0.05 to about 2-fold the number of carbohydrate repeat units in the polysaccharide.

In one embodiment, the polysaccharide-based nanoparticle is functionalized with 2-aminophenylboronic acid with molar equivalents of about 0.05 to about 2-2 fold the number of carbohydrate repeat units in the polysaccharide.

In one embodiment, cationic polysaccharide-based nanoparticles are functionalized with 4-bromobenzaldehyde via a condensation reaction with hydroxyl groups on the anhydrous glucose repeat unit with molar equivalents of about 0.05 to 2-fold the number of carbohydrate repeat units in the polysaccharide.

In another embodiment the total concentration of the polymer precursor(s) ranges from about 1 to about 50 w/v %, or from about 2 to about 35 w/v %, or about 5 to about 25 w/v %.

In one embodiment, the polymer precursor(s) are functionalized with functional groups including but are not limited to aldehydes, bromobenzaldehydes, or derivative thereof, carboxylic acids, amino groups, phenylboronic acid groups, phosphate groups, sulfate groups, zwitterionic groups, or hydrophobic moieties.

In one embodiment, the degree of functionalization of the polymer precursor(s) is quantified as about 1% to about 70 mol % of the total number of monomers on the polymer(s), or about 2% to about 50 mol % of the total number of monomers on the polymer(s), or about 5% to about 30 mol % of the total number of monomers on the polymer(s).

In one embodiment, the polymer precursor(s) are functionalized with two or more different functional groups including but not limited to functionalizing or copolymerizing different monomer groups within the polymer or copolymer or functionalizing a single monomer residue with two or more different functional groups.

In one embodiment, polymer precursors of two or more types or degrees of functionalization are used to prepare a single hydrogel, including but limited to combining different concentrations of different polymers varying in their degree of functionalization or functionalization moiety.

In one embodiment, at least one polymer precursor comprises of a cationic polymer with a zeta potential in the range of about +5 mV to about +50 mV or a anionic polymer with a zeta potential in the range of about −5 mV to about −50 mV. In a further embodiment, these polymers could be additionally functionalized. An example includes but is not limited to creating a negatively charged poly[oligo(ethylene glycol) methyl ether methacrylate]-hydrazine-dimethylmaleic acid copolymer.

In one embodiment, the polymer precursor(s) solution is mixed with an additive that alters the properties of the gelation process or the stability of the crosslinks, including but not limited to adding a gelator or an antioxidant.

In one embodiment, at least one polymer precursor is chitosan, carboxymethyl chitosan, or another chitin derivative. In an embodiment, the polymer precursor is carboxymethyl chitosan. In a further embodiment, the polymer is mixed with an additive such as ascorbic acid to modify the precursor solution pH.

In one embodiment, at least one polymer precursor is a co-polymer of poly[oligo(ethylene glycol) methyl ether methacrylate]-hydrazide-dimethylmaleic acid or one of its functionalized polymer derivatives.

In another embodiment, at least one polymer precursor is a glycosaminoglycan including but not limited to chondroitin sulfate A, chondroitin sulfate B, chondroitin sulfate C, hyaluronic acid, heparan sulfate, heparin, keratan sulfate, and their salts and their derivatives. The total concentration of glycosaminoglycan in the aqueous dispersion may range from about 2 w/v % to about 50 w/v % or about 5 w/v % to about 25 w/v %.

In another embodiment, at least one polymer precursor is functionalized with thiol groups. In an embodiment, a thiol-containing nucleophile is used to functionalize an electrophilic group on a glycosaminoglycan. For example, the amino group on cysteamine (nucleophile) can bind to a carboxy group on the glycosaminoglycan (electrophile) via carbodiimide coupling. In another embodiment, a thiol-containing electrophile is used to functionalize a nucleophilic group on the glycosaminoglycan. As a non-limiting example, an amino group on the glycosaminoglycan (nucleophile) can bind to the carboxy group on mercaptopropionic acid (electrophile) via carbodiimide coupling. In another embodiment, thiol functionalization is performed by reducing a pre-existing sulfate or sulfonate group on the glycosaminoglycan to a thiol.

In one embodiment, a gelator precursor is used to catalyze disulfide formation between thiol functionalized glycosaminoglycans to enable more rapid crosslinking. Examples of such gelators include but are not limited to oxidized glutathione. In one embodiment, the gelator is added in solution at a concentration ranging from about 1 to about 50 mM within the secondary polymer precursor solution. In another embodiment, the gelator precursor is functionalized onto the backbone of the glycosaminoglycan backbone. In one embodiment, 2,2′-dithiopyridine can functionalized onto thiol-functionalized glycosaminoglycan to be available for rapid disulfide exchange with other thiolated glycosaminoglycans.

In an embodiment, hydrogel gelation between the two precursors will occur inside a water-in-oil emulsion that templates the formation of a nanocluster nanogel. The emulsion may be a standard emulsion, a miniemulsion, a microemulsion, a nanoemulsion or any other form in which a water droplet can be stabilized inside an oil phase. In an embodiment, a miniemulsion is used. The oil phase may be any oil with a hydrophilic-lipophilic balance between about 0 to about 10, including but not limited to chloroform, hexanes, cyclohexane, dioxane, dimethyl sulfoxide, toluene, benzene, dichloromethane, acetophenone, pyridine, tetrahydrofuran, dioxane, silicone oil, olive oil, vegetable oil, canola oil, sesame oil, sunflower oil, fractionated coconut oil, or other organic oils.

In an embodiment, the volume of the hydrophobic phase is in excess to the volume of the aqueous phase. In a typical embodiment, the volume of the oil phase ranges from about 2 to about 200 times the volume of the aqueous phase, to about 5 to about 100 times the volume of the aqueous phase, or to about 10 to about 50 times the volume of the aqueous phase.

In an embodiment, low HLB value emulsifiers are used to stabilize the aqueous phase. These emulsifiers include but are not limited to mono- and di-glycerides, polyglycerol esters, sorbitan esters, polysorbates, ethoxylated mono- and di-glycerides, and corresponding blends. In an embodiment, the combination of any of these emulsifiers has an HLB value ±2 of that of the oil phase.

In another embodiment, high HLB value emulsifiers are used to stabilize the aqueous phase. These emulsifiers include but are not limited to mono- and di-glycerides, polyglycerol esters, sorbitan esters, polysorbates, ethoxylated mono- and di-glycerides, polyols and corresponding blends. In an embodiment, the combination of any of these emulsifiers has an HLB value ±2 of that of the aqueous phase.

In another embodiment, the total volume of emulsifiers is a fraction of the total volume of the corresponding phase, ranging from about 0.05 to about 20 v/v % of the total volume of the corresponding phase to about 0.1 to about 10 v/v % of the total volume of the corresponding phase to about 0.5 to about 5 v/v % of the total volume of the corresponding phase.

In an embodiment, energy must be added to the inverse miniemulsion to induce nanoscale droplets within the oil phase. Energy may be added to the system through a range of techniques including homogenization, bath sonication, probe sonication, mechanical stirring, magnetic mixing, membrane emulsification, and pressurized microfluidic mixing. In an embodiment, the total energy added to the system ranges from 1000 to 100000 J.

In another embodiment, the disclosure includes a hydrogel composition, comprising
a. at least one first precursor solution containing one or more types of a polysaccharide-based nanoparticle and
b. at least one second precursor solution containing a complementary polymer precursor that can induce gel formation.

In another embodiment, the polysaccharide-based nanoparticle contains or is functionalized with a functional group that can enable crosslinking with the complementary polymer.

In a further embodiment, the hydrogel is a bulk hydrogel.

In another embodiment, gelation is performed inside a water-in-oil emulsion that can template the formation of a microgel or nanogel.

In a further embodiment, sonication, homogenization, microfluidization, or other forms of mixing are used to form an emulsion, microemulsion, miniemulsion, nanoemulsion, or the like to template the formation of a microgel or nanogel.

In another embodiment, the crosslinking is reversible over time and/or in response to one or more environmental stimuli, including but not limited to pH, temperature, ionic strength, or the concentration of a particular chemical.

In another embodiment, the hydrogel crosslinks responsively degrade upon interaction with one or more specific biological environments, including
a. cellular or otherwise biological pH levels, including but not limited to those found in tumor microenvironments, or
b. cellular or otherwise biological enzyme concentrations, including but not limited to thioredoxin and glutaredoxin, or
c. cellular or otherwise biological condition concentrations of other molecules symptomatic of disease, including but not limited to increased glutathione levels.

In another embodiment, the crosslinking is via ionic interactions that can be reversed upon adjusting the pH from physiological pH to a pH relevant for site-specific biological delivery.

In another embodiment, at least one polymer contains a dimethylmaleic acid functional group

In another embodiment, there is included a hydrogel composition comprising
a. a polysaccharide-based nanoparticle functionalized with a first functional moiety; and
b. one or more polymers functionalized with a second functional moiety, wherein the first functional moiety and the second functional moiety are crosslinked through reversible covalent and/or physical crosslinks to form the hydrogel composition.

In another embodiment, the disclosure includes a hydrogel composition, comprising
a. at least one polysaccharide-based nanoparticle functionalized with one or more first functional moieties; and
b. at least one polymer functionalized with one or more second functional moieties, wherein at least one of the first functional moieties and at least one of the second functional moieties are crosslinked through covalent and/or physical crosslinks to form the hydrogel composition.

In one embodiment, the hydrogel composition comprises two or more polysaccharide-based nanoparticles which are functionalized with one or more first functional moieties.

In another embodiment, the hydrogel composition comprises a polysaccharide-based nanoparticle having at least two first functional moieties.

In one embodiment, the polysaccharide-based nanoparticle comprises starch, glycogen, cellulose, chitin, galactogen, arabinoxylans, pectins, pullulans, dextrans, chondroitin sulfates, hyaluronans, keratans, and derivatives and combinations thereof.

In one embodiment, the formed particles are starch with a particle diameter of less than 1000 nm, or less than 100 nm, or less than 50 nm, or less than 25 nm, or less than 20 nm, or less than 15 nm and exterior hydroxyl groups which can be chemically modified as desired. One embodiment of a starch nanoparticle has been described previously in European Patent EP 2 714 794 B1, described as “a curable composition, or binder. In one embodiment, the composition includes a dispersion in water, optionally a latex, of particles comprising a biopolymer. Optionally, the particles may comprise a) particles comprising crosslinked biopolymers, b) particles having an average size of less than 400 nm, c) particles having a volume swell ratio of 2 or more or d) particles comprising starch. The composition may also include a crosslinking agent, in addition to any crosslinking agent that may have been previously used to make the particle.

In another embodiment, each dimension of the hydrogel is greater than about 1 mm.

In another embodiment, the hydrogel is formed as a microparticle with at least one dimension less than one millimetre, a nanoparticle with at least one dimension less than one micrometre, or another kind of particulate form.

In one embodiment, the sizes of the polysaccharide-based nanoparticle and the hydrogel particle are selected to enable different biological responses.

In another embodiment, the polysaccharide nanoparticle is less than about 50 nm in, or less than about 25 nm in size to enable tissue penetration while the hydrogel particle is between 50 nm to 2000, optionally 100-500 nm in size to allow for circulation.

In one embodiment, the one or more polymers comprise starch, glycogen, cellulose, chitin, galactogen, arabinoxylans, pectins, pullulans, dextrans, chondroitin sulfates, hyaluronans, keratans, proteins, polynucleotides, or synthetic polymers, and derivatives and combinations thereof.

In another embodiment, the synthetic polymers comprise methacrylates, acrylates, esters, ethylene glycols, acrylamides, styrenes, cyanates, vinyl chlorides, siloxanes, silanes, urethanes, terephtalates, and combinations and derivatives thereof.

In another embodiment, the first functional moiety is a nucleophilic moiety and the second functional moiety is an electrophilic moiety. Nucleophiles include but are not limited to amines, thiols, alcohols, hydrazides, amides, azides, β-diketones, acetylides, cyanides, hydroxy succinimides, carboxylates, activated phenyls, and their derivatives and ions. Electrophiles include but are not limited to aldehydes, esters, anhydrides, acyl halides, β-unsaturated carbonyls, maleimides, succinimides, epoxides, lactones, lactams, carbamates, carbonates, benzyl halides, carbodiimides, peroxides, vinyl sulfones, and their derivatives and ions.

In a further embodiment, the first functional moiety is an electrophilic moiety and the second functional moiety is a nucleophilic moiety. Electrophiles include but are not limited to aldehydes, esters, anhydrides, acyl halides, β-unsaturated carbonyls, maleimides, succinimides, epoxides, lactones, lactams, carbamates, carbonates, benzyl halides, carbodiimides, peroxides, vinyl sulfones, and their derivatives and ions. Nucleophiles include but are not limited to amines, thiols, alcohols, hydrazides, amides, azides, β-diketones, acetylides, cyanides, hydroxy succinimides, carboxylates, activated phenyls, and their derivatives and ions.

In another embodiment, the covalent crosslinks include but are not limited to Schiff bases (i.e. imines), amides, disulfides, hydrazones, esters, thioesters, thioacetals, enamines, ethers, thioethers, sulfonamides, Diels-Alder linkages, Michael addition products, ureas, carbamates, carbonates, O-acylisoureas, β-thiosulfonyls, N-acylimidazioles, lactones, azolactones, and derivatives thereof.

In one embodiment, a covalent crosslink of the present disclosure comprises a disulfide bond formed between adjacent thiol groups optionally in the presence of a gelator. Alternately, in another embodiment, a non-covalent crosslink of the present disclosure comprises an electrostatic attraction between the negatively charged carboxyl groups of poly[oligo(ethylene glycol) methyl ether methacrylate]-hydrazine-dimethylmaleic acid copolymer and positively charged cationic starch nanoparticles.

In another embodiment, the physical crosslink includes but is not limited to electrostatic interactions, ionic interactions, pi-pi stacking, physical entanglements, hydrogen bonding, dipole-dipole interactions, van der Waals' forces, host-guest interactions, hydrophobic interactions, and combinations thereof.

In a further embodiment, the first functional moiety may include but is not limited to an aldehyde or derivative thereof, a sulfide, a carboxylic acid, an amino group, phenylboronic acid, a cationic group, an anionic group, and/or a hydrophobic moiety

In another embodiment, the aldehyde moiety is a bromobenzaldehyde moiety.

In another embodiment, the polysaccharide-based nanoparticle is a starch-based nanoparticle.

In another embodiment, the second functional moiety is an aldehyde or derivative thereof, a thiol, a carboxylic acid, an amino group, phenylboronic acid, a cationic group, an anionic group, a hydrophobic moiety, or a combination thereof.

In another embodiment, the hydrogel composition comprises
a. a starch-based nanoparticle functionalized with aldehyde groups; and
b. chitosan, carboxymethyl chitosan, or another chitosan derivative wherein the hydrogel is formed from reversible imine bonds.

In another embodiment, the hydrogel composition comprises
a. a starch-based nanoparticle functionalized with thiol groups;
b. chondroitin sulfate functionalized with thiol groups, wherein the hydrogel is formed from reversible disulfide bonds.

In another embodiment, the hydrogel composition comprises
a. a cationic starch-based nanoparticle functionalized with aldehyde groups;
b. a poly[oligo(ethylene glycol) methyl ether methacrylate] functionalized with carboxylic acid groups, wherein the hydrogel is formed from cationic-anionic interactions.

In another embodiment, the crosslinking is reversible over time and/or in response to one or more environmental stimuli, including but not limited to pH, temperature, ionic strength, or the concentration of a particular chemical.

In another embodiment, the polymer is a thiolated glycosaminoglycan.

In another embodiment, the thiolated glycosaminoglycan polymer is chondroitin sulfate A, chondroitin sulfate B, chondroitin sulfate C, hyaluronic acid, heparan sulfate, heparin, keratan sulfate, or salts and derivatives thereof.

In an embodiment, the polysaccharide-based nanoparticle and/or complementary polymer precursor are mixed with a crosslinking aid such as a gelator such as oxidized glutathione.

The present disclosure also includes a method for the administration of a hydrogel composition of the disclosure containing a medicine for the treatment of a condition, the method comprising co-administering to a patient a solution of the polysaccharide-based nanoparticle and a solution of the polymer for the in situ formation of the hydrogel composition in the patient.

In another embodiment, the solutions are for intravenous, intramuscular, intracranial, subcutaneous, intradermal, or intranasal administration.

In another embodiment, the solutions are administered with a double barreled syringe.

In another embodiment, the solutions contain microgels, nanogels, or other particulate hydrogels that can be injected using a single barrel syringe.

In another embodiment, the hydrogel is used to physically or chemically encapsulate proteins, cells, enzymes, drugs molecules or other therapeutics for the treatment of a condition.

In an embodiment, the hydrogel is loaded with a drug used as a therapeutic for schizophrenia, Parkinson's disease, bipolar disorder, or other neurological diseases.

In an embodiment, the hydrogel is loaded with (3R)-2-oxo-3-[[(2S)-2-pyrrolidinylcarbonyl]amino]-1-pyrrolidineacetamide) (PAOPA), prolyl-leucyl-glycinamide, haloperidol, loxapine, chloropromazine, perphenazine, clozapine, olanzapine, risperidone, lurasidone, ziprasidone, levodopa, carbidopa, benserazide, lithium, or other drugs targeting neurological conditions.

In one embodiment, the therapeutic agent is a drug, a protein, an antibody, an enzyme, a peptide, a polynucleotide such as DNA, RNA or aptamers, a carbohydrate, a glycoproteins or proteoglycan, or another molecule with relevant bioactivity.

In one embodiment, the therapeutic agent is used for the treatment of schizophrenia, Parkinson's disease, bipolar disorder, other neurological diseases, or cancer

In an embodiment, the hydrogel is used as a therapeutic for cancer.

In an embodiment, the hydrogel is loaded with doxorubicin, camptothecin, paclitaxel, sunitinib, cisplatin, or another chemotherapeutic drug.

In one embodiment, the crosslinking of the hydrogel is selected such that the kinetics of the decrosslinking reaction is increased in the cell environment in which the drug is intended to treat. For example, in one embodiment, when the condition is cancer, disulfide crosslinking is utilized as cancer cells are rich in glutathione, a disulfide-reducing tripeptide which is overly expressed in cancer cells. In one embodiment, the kinetics of the reversible crosslinking (decrosslinking) of the disulfide bond is increased when administered at the site of the cancer cells. In another embodiment, one or more precursor solutions are functionalized with a functional group intended to stabilize an encapsulated therapeutic.

In one embodiment, the hydrogel composition comprises one or more different polysaccharide-based nanoparticles functionalized with different functional groups to deliver different therapeutic agents. In one embodiment, the kinetics of the different crosslinking groups allows for the delivery of different drugs at different times based on the decrosslinking reaction of the different crosslinks.

In another embodiment, one or more precursor solutions are functionalized with a functional group intended to alter the drug loading and/or release of an encapsulated therapeutic.

In another embodiment, phenylboronic acid functional groups are used to stabilize drugs with cis-diol functional groups.

The following non-limiting examples are illustrative of the present applications of the hydrogels of the present disclosure.

### EXAMPLES

### Example A1: Synthesis and Physicochemical Characterization of Functionalized Starch Nanoparticles and Hydrogel Prepolymers

### Materials

Experimental grade unfunctionalized starch nanoparticles (SNP) were obtained from EcoSynthetix Inc. and were dialyzed against DIW for a minimum of 6 (6+ hour) cycles before use. O-Carboxymethyl chitosan was obtained directly from Bonding Chemical (Katy, Tex., USA) with >80% degree of functionalization. (3R)-2-Oxo-3-[[(2S)-2-pyrrolidinylcarbonyl]amino]-1-pyrrolidineacetamide) (PAOPA) was purchased from Tocris Bioscience (Oakville, ON, Canada). 2-Aminophenylboronic acid (2-APBA, 97%) was purchased from Fisher Scientific. Polyglycerol polyricinoleate (PGPR) 5175 was obtained from Paalsgaard (Morris Plains, N.J., USA). Fractionated coconut oil 100% MCT oil was obtained from Voyageur Soap & Candle Co. N′-ethyl-n-(3-dimethylaminopropyl)-carbodiimide (EDC) was obtained from Carbosynth. Carboxymethyl chitosan oligosaccharide lactate (COL), (+)-5-methyl-10,11-dihydro-5H-dibenzo [a,d]cyclohepten-5,10-imine maleate salt, ((+)-MK-801), 6-hydroxydopamine hydrochloride (6-OHDA, 97%), sodium periodate (NaIO4, 99%), diethylene glycol, chloroacetic acid (99%), Span® 80, hydroxylamine hydrochloride, and 3,4-dihydroxy-L-phenylalanine (levodopa, L-DOPA, 97%) were all obtained from Millipore Sigma and were used as received. 2-ethanesulfonic hemisodium salt (MES) was purchased from Millipore Sigma and added to MQW to make a 0.1M solution at 6.8 pH. Phosphate buffered saline solutions were made from tablets (PBS; contains 10 mM phosphate buffer, 140 mM NaCl, 3 mM KCl, and pH 7.4 when dissolved in 200 mL water, Millipore Sigma). For all experiments, Milli-Q water was used.

SHSY-5Y human neuroblastoma cells were obtained from American Type Culture Collection (ATCC). Human nasal epithelial cells (hNEpC), Airway Epithelial Cell Medium and Growth Medium Supplement Mix were purchased from PromoCell (Heidelberg, Germany). Dulbecco's Modified Eagle Medium: Nutrient Mixture (DMEM:F12) media, L-glutamine, and penicillin streptomycin we purchased from ThermoFisher Scientific (Burlington, ON, Canada). Resazurin dye solution and toxicology assay kit was obtained from Sigma-Aldrich (Oakville, ON, Canada). 3(4,5-dimethylthiazol-2-yl)-2,5diphenyl-2H-tetrazolium bromide (MTT). Isoflurane was obtained from CDMV (Saint-Hyacinthe, QC, Canada). Male Sprague-Dawley rats were obtained from Charles River at 250-300 g (St. Constant, QC, Canada).

LMA MAD internasal atomizer devices were obtained from Equipment Medical Rive Nord (Montreal, QC, Canada). L-series double barrel syringes were obtained from MEDMIX (Rotreux, Switzerland).

Synthesis of Aldehyde functionalized Starch Nanoparticles (SNP-CHO)

Starch nanoparticles (SNP) were dialyzed against DIW for a minimum of 6 (6+ hour) cycles before use. SNPs were subsequently functionalized using sodium periodate in order to substitute the C2 and C3 cis hydroxyl groups on the glucose monomeric backbone with aldehyde groups. 10 g of dry SNP (post % solids determination) was dispersed in 400 mL of Milli Q water (MQW), and the pH was adjusted to neutral using 10 mM HCl or 10 mM NaOH. A second solution of sodium periodate (3.3 g for 0.25 eq and 6.6 g for 0.5 eq of SNP) was dissolved in 100 mL of MQW. The contents of solution 2 were added to that of solution 1 slowly and then allowed to react at room temperature (RT˜25° C.) for 18 hours. The reaction vessel containing the starch nanoparticles and sodium periodate was covered with aluminum foil to exclude light for better reaction efficiency. The reaction was terminated with ethylene glycol (1.47 mL for 0.25 eq and 2.94 mL for 0.5 eq), after which the product was then dialyzed (6×6 h cycles, 3.5 kDa molecular weight cut-off membrane), lyophilized, and stored as a dry powder on the benchtop.

Synthesis of 2-Aminophenylboronic Acid functionalized Starch Nanoparticles (SNP-APBA)

SNP-CHO (5 g) was dispersed in 100 mL MQW and brought to pH of 10 with 0.1M NaOH at 50° C. 20 g of chloroacetic acid was added to 50 mL MWQ and then subsequently added into the SNP-CHO solution and left to react for 4 hours. The reaction was terminated by returning the pH to 7 with 1 M HCl. The carboxylic acid functionalized SNP product (SNP—COOH) was then dialyzed (6×6 h cycles, 3.5 kDa molecular weight cut-off membrane), lyophilized, and stored as a dry powder on the benchtop.

SNP—COOH (1 g) was dispersed in 100 mL MQW. 0.27 g of 1-ethyl-3-(3-dimethylaminopropyl)-carbodiimide (EDC) was added and the solution was brought to pH of 4.75. 0.25 g of 2-aminophenylboronic acid (2-APBA) was then added dropwise into the solution, and the reaction was held at a pH of 4.75 for 24 hours. The reaction was terminated by returning the pH to 7 with 1M NaOH. The 2-aminophenylboronic acid functionalized SNP product (SNP-APBA) was then dialyzed (6×6 h cycles, 3.5 kDa molecular weight cut-off membrane), lyophilized, and stored as a dry powder on the benchtop.

### Characterization

### Determining the Degree of Aldehyde Substitution of SNP-CHO

The degree of substitution of SNP-CHO was quantified using colorimetric base-into-acid titration, while the degree of substitution (DS) of free aldehydes was determined reacting free aldehydes with hydroxylamine salt to generate a titratable strong acid.

SNP-CHO nanoparticles were dispersed in 50 mL of a pH 4, 0.25M hydroxylamine hydrochloride solution with a concentration at 50 mg/mL (1 w/v %), vortexed (1600 rpm) for 1 minute, and placed into a water bath sonicator for 5-10 min. Vortexed samples were filtered with a 0.45 m PTFE syringe filter to remove any particle aggregates. Filtered nanoparticles were added to a glass 80 mL beaker, after which a base-acid titration was performed using 0.1M NaOH at a rate of approximately 10 min/PH unit. Functionalization was calculated by subtracting the degree of functionalization measured on base SNP (before oxidation) from the degree calculated in the oxidized nanoparticle. Values are presented in % functionalization per 100 AGU measured on the Burivar-I2 automatic buret (ManTech associates)

### Fourier Transform (ATR-FTTIR)

Attenuated total reflectance Fourier transform infrared spectroscopy (AT-FTIR) was conducted using a Vertex70 Platinum ATR FT-IR (Bruker, Billerica, Mass., USA; temperature=25° C.) to detect the appearance of aldehyde groups following oxidation, using 64 scans within the wavenumber range from 4000 to 350 cm⁻¹ with a resolution of 4 cm⁻¹. The SNP-CHO and CMCh samples tested were purified and dried using dialysis with 3.5 kDa MWCO bags (Spectrapor) for 6×6 h cycles and lyophilized prior to testing.

### Dynamic Light Scattering (DLS)

SNP-CHO nanoparticles were dispersed in H₂O with a concentration of 10 mg/mL (1 w/v %), vortexed (1600 rpm) for 1 minute and placed into a water bath sonicator for 5-10 min. Vortexed samples were filtered with a 0.45 m PTFE syringe filter to remove any large aggregates. Filtered clusters were added to a plastic (polypropylene) cuvette and measured on a NanoBrook 90Plus PALS (Brookhaven, Long Island, N.Y., USA; temperature=25° C.). Zeta potential was measured in the same way.

### Nanoparticle Tracking Analysis (NTA)

Nanoparticle tracking analysis (NTA, NanoSight LM10) was used to measure the number particle size distribution for certain formulations. All samples were run at a concentration of 100 g/mL in MQW, sonicated for 5-10 min in a bath sonicator and passed through a 0.45 m PTFE syringe filter to remove large aggregates before testing, as described for DLS measurements. Single nanoparticle tracking analysis (NTA 3.4) was analyzed with a LM14 HS NanoSight microscope (Malvern Panalytical, Worcester, UK; 100 to 200 particles per frame).

### Transmission Electron Microscopy (TEM)

Transmission electron microscopy (TEM) was performed using a JEOL 1200EX TEMSCAM instrument to assess particle size and structure. In order to prevent film formation of SNPs on the TEM grid, a low contrast poly(methyl methacrylate) (PMMA) latex (particle size=300 nm) was used as an imaging aid by aliquoting 0.5 w/v % SNPs with 0.1 w/v % PMMA latex dispersions on a standard carbon/Formvar TEM grid and drying overnight prior to measurement. Average particle size results are reported based on the average of the sizes reported from ImageJ analysis of 30-40 particles, with the error bar representing the standard deviation.

### Results and Discussion

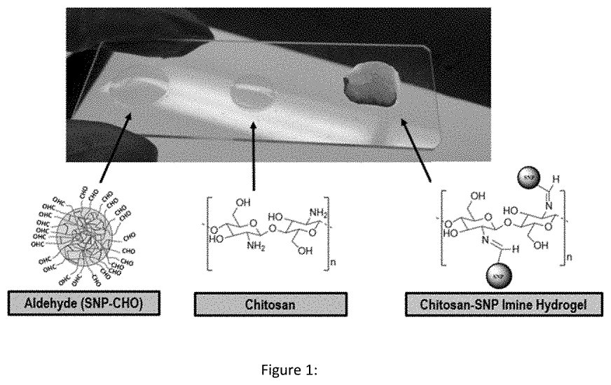

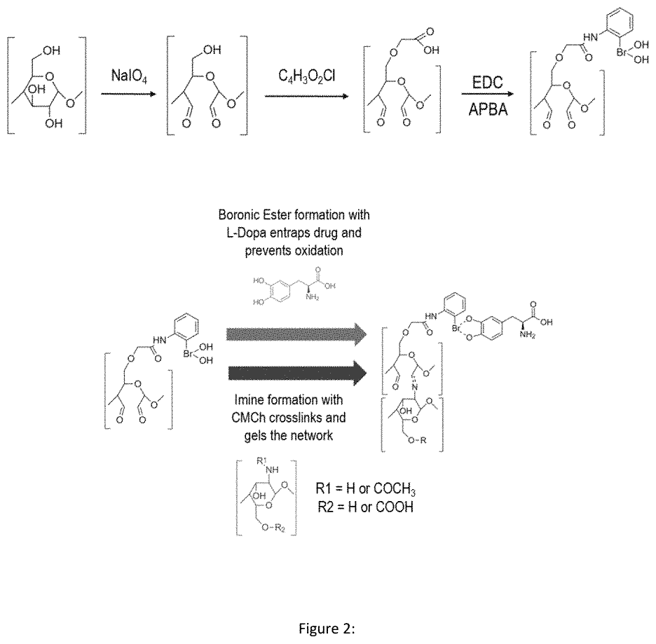

SNPs were chemically functionalized with aldehyde groups for enabling Schiff base crosslinking with amine-containing secondary precursor polymers (FIGS. 1, 2). The aldehyde content was quantified via conductometric titration after reacting with hydroxylamine, yielding an aldehyde DS (degree of substitution) of 0.24. Oxidation of the C2/C3 cis-diol groups on the anhydrous glucose unit of starch to form aldehyde groups was confirmed qualitatively via ATR-FTIR spectroscopy, with an aldehyde peak visible at 1800 cm⁻¹. ¹H-NMR confirms the generation of SNP-CHO with degrees of substitution ranging between 0.25-0.5 depending on the amount of sodium periodate added to the reaction. Varying the aldehyde DS on the precursor can change the number of potential crosslinks in the hydrogel and thus the hydrogel mechanical and swelling properties.

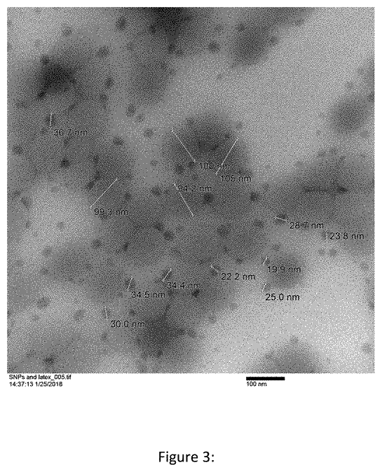

Dynamic light scattering indicated that the major fraction of starch nanoparticles had a size of 20-50 nm. Transmission electron microscopy confirmed the average size of the SNP-CHO particles as being between 20-40 nm and the shape to be largely spherical, with any observed shape deformation likely attributable to the drying of a soft gel-like nanoparticle in the presence of the hard anti-film forming PMMA latex (visible as the lighter larger particles in the background of the TEM image, FIG. 3). The zeta potential of the SNPs was −15 to +15 mV, confirming the maintained neutral charge.

### Example A2: Synthesis and Physicochemical Characterization of Functionalized Starch Nanoparticle and Carboxymethyl Chitosan Hydrogels

### Preparation of Starch Nanoparticle and Carboxymethyl Chitosan Precursor Solutions

The functionalized starch nanoparticle hydrogel precursor was created by manually agitating (5-20,000 rpm) a SNP-CHO suspension at a concentration range of 3-35 w/v % in Milli-Q water (MQW).

The carboxymethyl chitosan (or oligosaccharide formulation (COL)) hydrogel precursor was created by manually agitating (5-20,000 rpm) CMCh or COL at concentration range of 2-6 w/v % in MQW.

### Synthesis of Starch Nanoparticle and Carboxymethyl Chitosan (SNP-CHO@CMCh) Bulk Hydrogels

200 or 300 μL of SNP-CHO solution was pipetted into a silicone mold of a defined volume (400 or 600 μL total), followed by the subsequent addition of an equal volume of CMCh solution (200 or 300 μL) to the same mold. Alternatively, the two precursor materials can be loaded into a double barrel syringe and ejected through a needle tip to allow for homogenous mixing. The mold is allowed to sit at room temperature for a defined time (24 h), after which the gelled product is manually removed from the silicone mold.

In an embodiment, the SNP-CHO solution can be replaced with SNP-APBA of the same w/v %. The gel forms using the same principles but will contain the additional APBA functionalization.

In an embodiment, one or both of the precursor solutions can be mixed with a drug. In one embodiment, the drug is the anti-schizophrenic drug PAOPA added at a concentration of 1.66 mg/mL.

### Synthesis of Starch Nanoparticle and Carboxymethyl Chitosan (SNP-CHO@CMCh) Nanogels

A 3 w/v % suspension of SNP-CHO in H₂O and a 2 w/v % solution of CMCh in H₂O were prepared. Meanwhile, a 50 mL Falcon tube containing fractionated coconut oil (25 or 30 mL), Span® 80 (1 or 2 mL) and PGPR (0.25 or 0.5 mL) formed the oil phase by mixing at 300 rpm for 15 minutes. Following, 0.5 mL of each SNP—CHO and CMCh precursor solutions were loaded into a double-barrel syringe and the solution was depressed slowly into the oil phase dropwise over the course of 1 minute while the oil phase was under 1200 rpm homogenization (Digital Ultra Turrax homogenizer, IKA (Staufen, Germany)). After 10 minutes of homogenization, the resulting emulsion was immediately placed in an ice bath and probe sonicated (Q700a sonicating probe (Qsonica, Newtown, Conn., USA)) at 50 W for 2 minutes. The SNP-CHO@CMCh nanogel emulsion was immediately transferred to a scintillation vial and cooled to room temperature on the benchtop (˜3-4 hrs). Following, the nanogels were purified using vortex centrifugation at 20,000 rpm for 10 min, after which the oil phase supernatant was discarded and the nanogel pellet was resuspended in water using a tabletop vortex.

### Characterization of the Bulk Hydrogel

### Gelation Kinetics

Gelation was validated by performing a vial inversion experiment to determine the time it takes for a precursor polymer combination to cease to flow when held upside down. In a 1.5 mL Eppendorf tube, 100 μL CMCh/COL (2-6 w/v %) and 100 μL of SNP-CHO (5-35 w/v %) were added and regularly inverted every 2 seconds. The gelation time (t_{gel}) was recorded as the time at which no flow was observed within 1 second after sample inversion. All gel combinations were run in triplicate (n=3).

### Degradation and Swelling of Bulk Hydrogel Disks in Vitro

The formed bulk hydrogels (400 or 600 μL) were loaded into pre-weighed 6 well-plate transwell inserts (VWR) to allow for free diffusion of water and immersed fully in 10 mM PBS (0.01 M phosphate buffer, pH 7.4, at 25° C.). At predetermined time intervals (every half hour for the first hour, every hour for the next 4 hours and then twice a day for two weeks), the transwell inserts were removed, excess water was wicked away using a Kimwipe, the sample mass was weighed, and the inserts were returned to the tray. The experiment continued until the hydrogels were completely degraded.

The formed bulk hydrogels (with or without loaded drug) (400 or 600 μL total volume) were loaded into pre-weighed 6 well-plate transwell inserts (VWR) to allow for free diffusion of water and immersed fully in 10 mM PBS (0.01 M phosphate buffer, pH 7.4, at 25° C.). At predetermined time intervals (every half hour for the first hour, every hour for the next 4 hours and then twice a day for two weeks), the transwell inserts were removed, excess water was wicked away using a Kimwipe, the sample mass was weighed, and the inserts were returned to the tray. The experiment continued until the hydrogels were completely degraded.

### Mechanical Testing of Bulk Hydrogel Disks

The mechanical properties of the bulk hydrogel disks were assessed using a MACH-1 Micromechanical Analyzer (Biomomentum, Inc., Laval, QC Canada). For all small angle oscillatory measurements, a strain sweep was performed from 0.1° to 2.154° at a frequency of 1 Hz to determine the linear viscoelastic region. Following, a frequency sweep was performed from 0.1 Hz to 2.5154 Hz within this linear viscoelastic regime (0.4-1.5 s⁻¹) to determine the storage modulus (G′) and loss modulus (G″) of the hydrogels.

### Mucoadhesion Testing of Bulk Hydrogel

PDMS Sylgard elastomers (dimensions 22×22 cm) were fabricated via manufacturer's instructions and dip-coated coated with 1% or 5% bovine mucin (Sigma Aldrich). The PDMS sheets were attached to an in-house designed lap-shear device attached to a MACH-1 micromechanical tester (Biomomentum Inc.) equipped with a 1 kg load cell. The work of adhesion was calculated based on the area under the curve using the trapezoidal method.

### Nebulization and In Situ Gelation Testing of Bulk Hydrogel

A double barrel syringe was filled with SNP-CHO and CMCh precursor solutions in either side of the device. An LMA intranasal mucosal atomization device was attached to the end of the double-barrel syringe to effectively aerosolize the gel precursors to form a sprayable hydrogel. Nebulization was tested at vertical distances ranging from 0-15 cm from the target application site.

### Characterization of the Nanogel

### Dynamic Light Scattering (DLS)

SNP-CHO@CMCh nanogels were dispersed in water at a concentration of 10 mg/mL (1 w/v %), vortexed (1600 rpm, 1 minute), and placed into a bath sonicator for 5-10 min to disperse the SNPs. Vortexed samples were filtered with a 0.45 m PTFE syringe filter to remove large aggregates. Filtered clusters were added to a plastic (polypropylene) cuvette and measured on a NanoBrook 90Plus instrument (Brookhaven, Long Island, N.Y., USA; temperature=25° C.).

### Nanoparticle Tracking Analysis (NTA)

SNP-CHO@CMCh nanogels were suspended at a concentration of 100 μg/mL in water, sonicated for 5-10 min in a bath sonicator, and passed through a 0.45 m PTFE syringe filter to remove large aggregates before testing. Single nanoparticle tracking analysis (NTA 3.4) was conducted with a LM14 HS NanoSight instrument (Malvern Panalytical, Worcester, UK; 100-200 particles per frame).

### Transmission Electron Microscopy (TEM)

Transmission electron microscopy (TEM) was performed using a JEOL 1200EX TEMSCAM instrument to access nanocluster size and structure. Images were captured of nanogels in PBS solution after 1 day and 10 days stored at 2 different temperatures.

### Results and Discussion

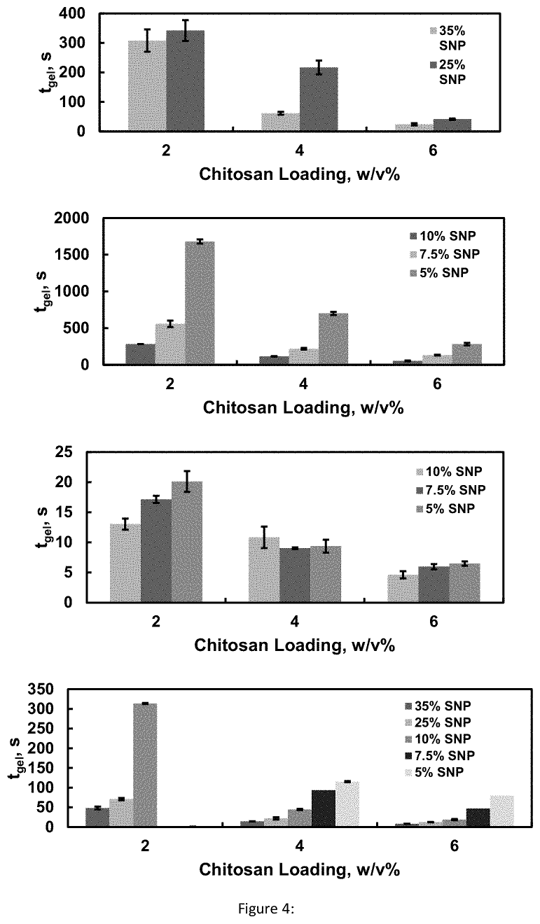

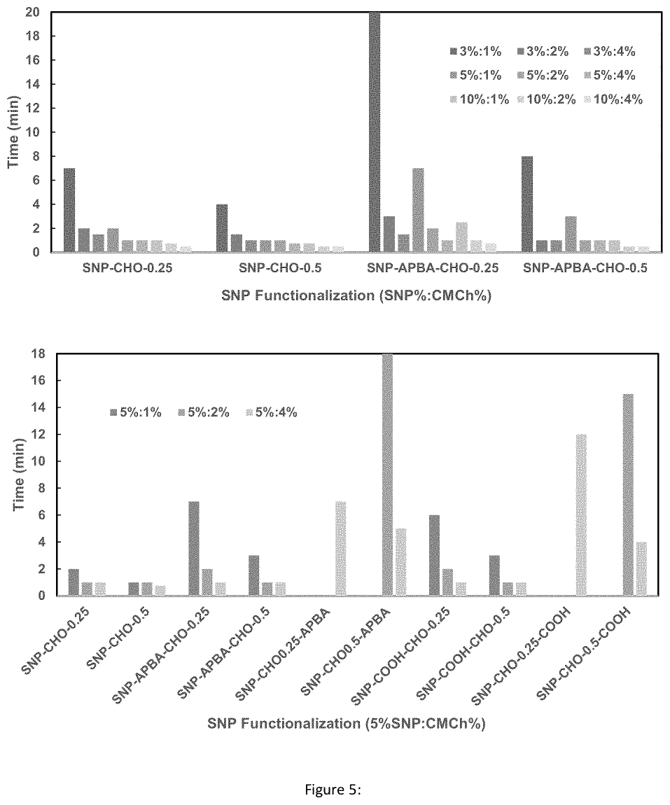

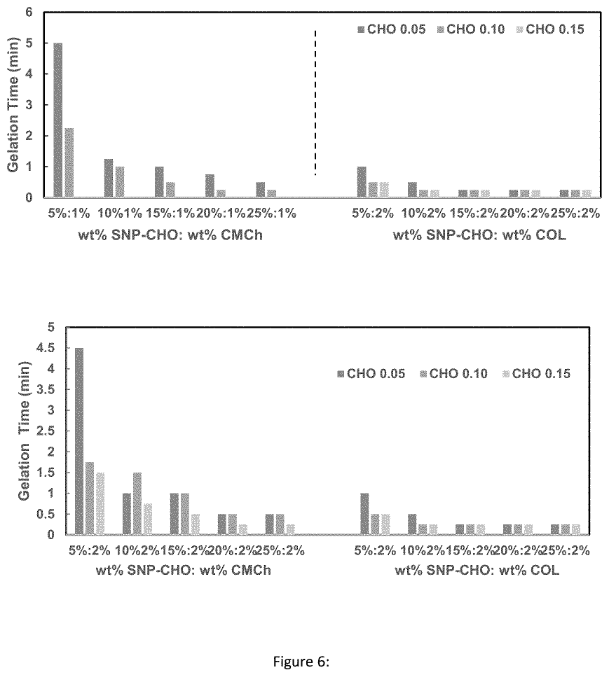

The fabrication of both SNP-CHO@CMCh and SNP-APBA@CMCh bulk gels proceeded via a similar mechanism of Schiff base formation. Physicochemical properties of the two gel morphologies are highly dependent on the w/v % of the precursor solutions as well as the DS of aldehyde groups on SNP-CHO. The gelation times observed following mixing of various concentrations of CMCh (2-6 w/v %, with the upper limit corresponding to the highest w/v % at which CMCh remained an injectable solution) with various concentrations and degrees of oxidation of SNP-CHO are shown in FIG. 4 and FIG. 5 and SNP@COL in FIG. 6. Note that the y-axis scales and the SNP concentrations tested for each aldehyde degree of substitution are significantly different, corresponding to the different concentrations of SNP required to make gels in each case and the different gelation times that result from those formulations. As the weight/volume percent (w/v %) of SNP-CHO or chitosan increased, gelation time decreased; correspondingly, increasing the functionality of SNP-CHO resulted in decreased gelation times. This result is consistent with the proposed Schiff base-induced crosslinking mechanism, in which the presence of more aldehyde and/or amine groups results in more crosslinking and thus faster gel formation. Hydrogels prepared with SNP-CHO-0.25 (DS of 0.25) required at least 25 w/v % SNPs to fabricate gels, while increasing the degree of oxidation of the SNPs (SNP-CHO-0.50 or SNP-CHO-1.0) resulted in gel formation within the <10 minute timeframe for SNP concentrations as low as 5 w/v %, with gels based on SNP-CHO-1.0 gelling consistently in <20 s. This accessible range of gelation times can be applicable to different applications. For example, given the practical considerations around performing in situ gelation in the nose, for which gelation times on the order of a few minutes are anticipated to be ideal to enable facile injection/spray but still promote good retention of the gel on the nasal mucosa, formulations based on SNP-CHO-0.25 enable rapid gelation with very high potential SNP loadings for enabling high drug concentrations.

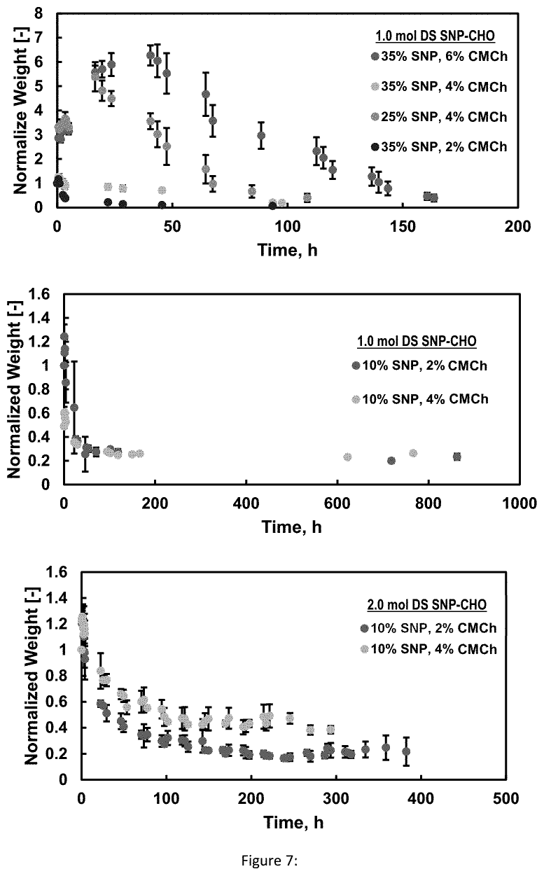

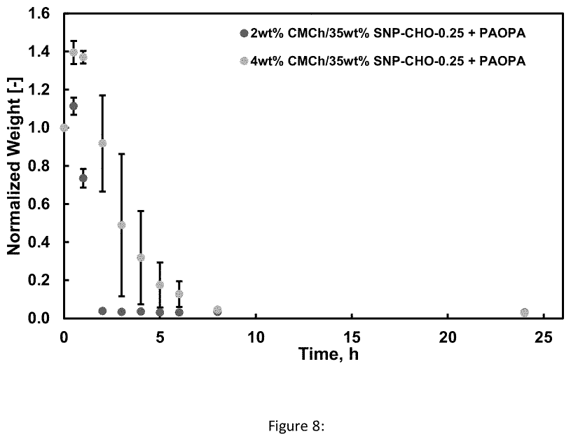

To assess the hydrolytic stability of the resulting labile Schiff base crosslinked hydrogels, the mass of the gels was tracked over time to gravimetrically assess the swelling and ultimate degradation of the hydrogel with and without drug (FIG. 7,8). Hydrogels with higher CMCh contents and lower SNP-CHO contents tended to exhibit substantial swelling upon incubation in PBS, with the 6 w/v % CMCh/35 w/v % SNP-CHO-0.25 hydrogel in particular exhibiting swelling ratios up to 6.5× its original mass prior to the onset of mass loss due to degradation. In contrast, all hydrogels prepared with SNP-CHO-1.0 and hydrogels prepared with SNP-CHO-0.25 but with higher SNP contents and lower CMCh contents (i.e. 4 w/v % CMCh/35 w/v % SNP-CHO-0.25 and 2 w/v % CMCh/35 w/v % SNP-CHO-0.25) exhibited relatively low swelling ratios. Hydrogels were observed to fully degrade in as little as 6.5 days (150 h) in PBS, with hydrogels prepared with SNP-CHO-0.25 showing predictably faster degradation times compared to more highly aldehyde-functionalized SNP-CHO-1.0 hydrogels. Hydrogel formulations loaded with 1.66 mg/mL PAOPA degrade significantly faster than their unloaded gel counterparts (FIG. 8), suggesting that the PAOPA peptide can competitively form Schiff base interactions with SNP-CHO and thus reduce the number of available Schiff base pairs available to form crosslinks.

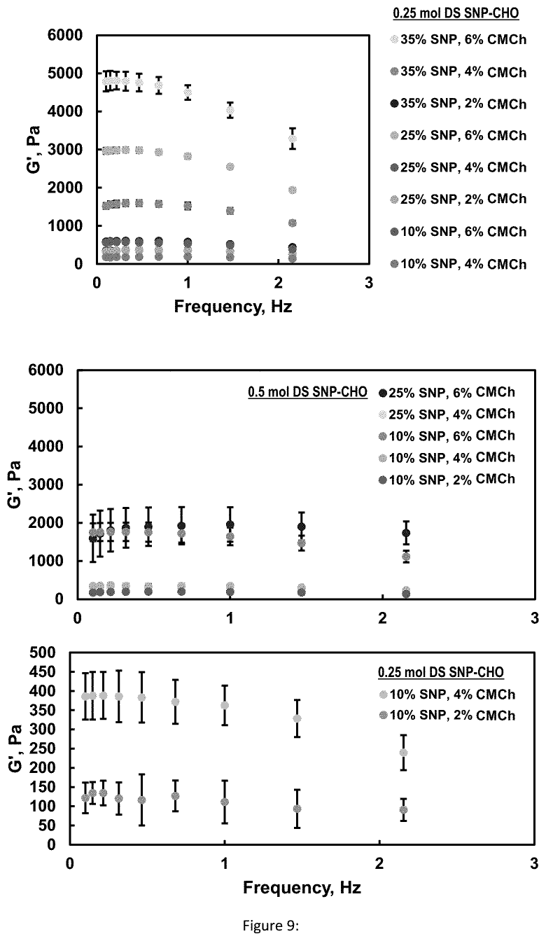

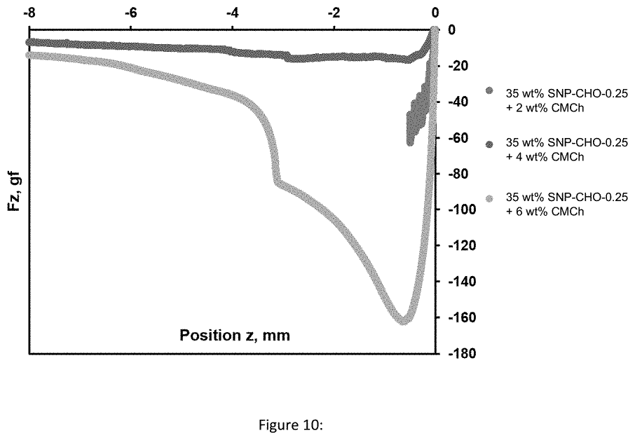

The shear storage moduli of the fabricated hydrogels are shown in FIG. 9. Increasing the concentration of CMCh or the concentration and/or degree of functionalization of SNP-CHO resulted in the formation of stiffer hydrogels. Most hydrogels showed G′ values of 2 kPa or less, except for gels prepared with 6 w/v % CMCh and higher SNP-CHO loadings. Given the low shear storage modulus of the nasal mucosa (˜30 Pa)³⁸, weaker hydrogels that still exhibit sufficiently fast gelation rates and prolonged degradation times are likely to better mechanically integrate with the native mucosa. This hypothesis was supported by measuring the mucoadhesion, (FIG. 10) with substantial interaction forces measured between the hydrogels and a model mucous-coated silicone sheet that increased as the concentration of the more mucoadhesive chitosan component of the hydrogels was increased (0.37 N mm for 35 w/v % SNP-CHO-0.25+2 w/v % CMCh, 1.05 N mm for 35 w/v % SNP-CHO-0.25+4 w/v % CMCh, and 5.12 N mm for 35 w/v % SNP-CHO-0.25+6 w/v % CMCh). Based on these results, formulations consisting of 2 or 4 w/v % CMCh mixed with 10-35 w/v % of SNP-CHO-0.25 or SNP-CHO-0.5 would result in hydrogels that can stick to the nasal mucosa, form in less than 10 minutes, and degrade in a week or less.

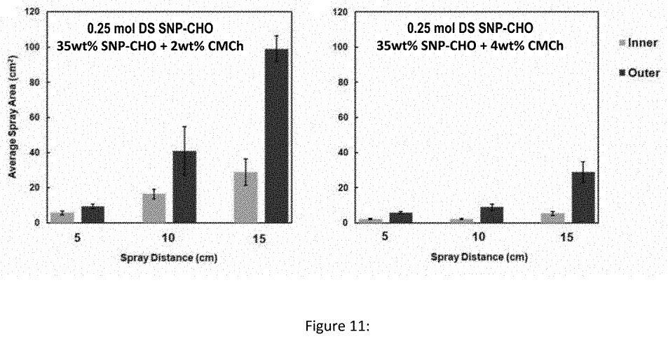

Nebulization of the hydrogel materials was characterized to determine the materials efficacy for intranasal applications. To confirm the ease of spray-based administration of the selected optimal formulations for intranasal delivery, the potential of the formulations to be nebulized was assessed using a nasal intranasal mucosal atomization device. FIG. 11 shows the average spray areas achieved as a function of the spray distance to a vertically-mounted paper substrate, with the maximum tested 15 cm spray distance corresponding to the maximum typical distance between the human nostril and nasopharynx. In both cases, an inner (thicker) and outer (thinner) layer was observed, with the former representing the focus of the spray and the latter representing the total potentially impacted surface following spray. At a distance of 5 cm, both the inner and outer profiles were relatively close, covering 6-10 cm²; these areas reasonably diverged at larger distances, with inner areas of −30 cm² and outer areas of up to ˜100 cm² observed at a 15 cm spray distance for 2 w/v % CMCh/35 w/v % SNP-CHO-0.25 (FIG. 11—Left). These areas are consistent with the surface area of the human intranasal cavity (˜80-160 cm²)³⁹, suggesting the potential for effective spray-based delivery of this formulation. The higher viscosity of the 4 w/v % CMCh/35 w/v % SNP-CHO-0.25 formulation resulted in smaller overall spray distances and less uniform nebulization (FIG. 11—Right), although the formulation could still be delivered via spray-based administration to form a less regular hydrogel film over a smaller surface area.

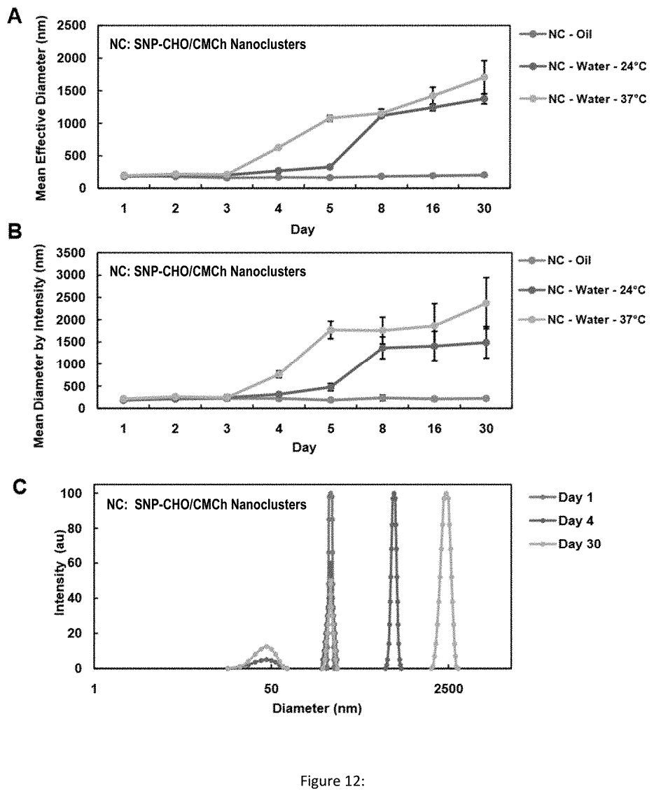

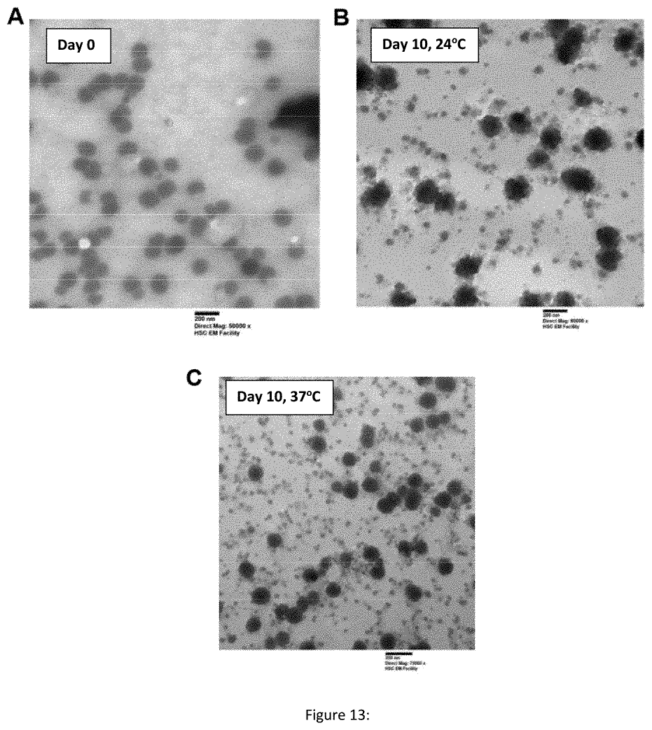

Dynamic light scattering (DLS) was used to track the degradation of the nanocluster nanogels into their precursor polymers over time. FIG. 12 shows the mean diameters of the nanocluster nanogels over time. The hydrodynamic diameter of the SNP@CMCh nanogel was determined to be 150-200 nm via DLS (FIG. 12A) and 184±50 nm via NTA (FIG. 12B). FIG. 12C depicts the resulting trimodal distribution of the nanocluster nanogels as they break down into their precursor materials over time. TEM shows a similar degradation profile, with SNPs clearly visible outside of the clusters indicating SNP release over a function of time as the nanocluster nanogels degrade (FIG. 13). These results indicate the nanocluster nanogels are capable of breaking down into their constituents and releasing SNPs over time.

### Example A3: Biological Applications of Functionalized Starch Nanoparticle and Carboxymethyl Chitosan Hydrogels

### In Vitro Cytotoxicity Testing

The cytocompatibility of the hydrogel precursor solutions was tested using SH-SY5Y human neuroblastoma cells and primary human nasal epithelial cells (HNEpC) cultured in media to ˜70% confluency. Following trypsin addition and centrifugation, 50 μL of a 200,000 cells/mL cell suspension (corresponding to 10,000 cells/well) were plated in a 96 well plate and incubated for 24 hours. SH-SY5Y human neuroblastoma cells were maintained in DMEM:F12 media supplemented with 10% fetal bovine serum and 1% penicillin-streptomycin (100 U/mL). Primary human nasal epithelial cells (HNEpC) were cultured in airway epithelial cell growth medium (Promo Cell) supplemented with 1% penicillin-streptomycin (100 U/mL). Cells were grown at 37° C. in a humid incubator (5% CO₂/95% air). Polymer solutions were prepared in sterile 10 mM PBS and filtered through a 0.2 μm syringe filter (SNP-CHO) or 0.8 μm syringe filter (CMCh, accounting for the higher viscosity of this solution) and added to the wells at concentrations between 0.1-10 mg/mL. After 24 hours of incubation, 50 μL of 3(4,5dimehtlythiazol-2-yl)-2,5diphenyl-2H-tetrazolium bromide (MTT) was added to each well as per the manufacturer's protocol. After another 24 hours of incubation, plates were read using an Infinite M200 Pro (Tecan) plate reader using an excitation of 560 nm and emission of 590 nm and gain of 70-80%. For all samples, the fluorescent intensities were related to positive (healthy cells) and negative (blank wells) controls to calculate the percent viability of cells for each concentration and material tested (n=4).

### In Vitro Drug Release of Antipsychotic Drugs

SNPs and CMCh were suspended/dissolved in an aCSF solution containing 1.66 mg/mL PAOPA and left to equilibrate for 24 hours, after which hydrogels were prepared as described to physically entrap PAOPA a model drug in the gel. Following overnight equilibration, the hydrogels were loaded into cell inserts and incubated in 10 mL of pH 6 PBS inside a 6 well plate at 37° C. and 100 rpm. At defined time points, the supernatant was sampled and the PAOPA drug release was assessed via gradient high performance liquid chromatography (HPLC, Waters, Milford, Mass., USA) using a Waters 1525 binary pump, a Waters 2707 autosampler, a C-18 column (5 μm, 150 mm×4.6 mm), and a Waters 2489 UV/visible detector (λ=215 nm for PAOPA) operating at ambient temperature and a flow rate of 1.0 mL/min. To elute PAOPA, a gradient mobile phase of 70% water/30% acetonitrile held constant for 5 minutes, gradually switching to 30% water/70% acetonitrile over the next 7 minutes, and then holding the composition constant for the next 5 minutes was used, after which the mobile phase was reverted to 70% water/30% acetonitrile and held constant for 15 minutes to recover the baseline. All samples were run in triplicate (n=3) for each gel formulation.

### In Vivo Schizophrenic Model Induction to Measure Delivery of Antipsychotic Drugs to the Brain

Age-matched male Sprague-Dawley rats (250-300 g, Charles River Canada, St. Constant, QC, Canada) received care that complied with protocols approved by the Animal Research Ethics Board at McMaster University and the guidelines of the Canadian Council on Animal Care. Animals were housed individually in standard cages on a reverse 12 h light cycle. Upon arrival, animals were habituated to their holding room for 1 week, followed by a week of handling (touching and petting for at least 5 minutes each, every other day) prior to any testing. Animals were handled regularly throughout the duration of all experimentation (2-3 times per week outside of experiments) and housed in a room maintained at 22° C. with 50% humidity and access to food and water ad libitum. The animal masses at the end of the experiments were between 400-550 g.

For the in situ animal injections, all drugs were dissolved in artificial cerebrospinal fluid (aCSF) solution formulated in-lab comprised of 120 mM NaCl, 4.8 mM KCl, 1.2 mM KH₂PO₄, 1.2 mM MgSO₄, 25 mM NaHCO₃, 2.5 mM CaCl₂), and 10 mM D-glucose. Further, PAOPA (with or without hydrogel formulation) was administered at concentration of 0.5 mg/kg intranasally, selected based on effective concentrations observed via dose-dependent intraperitoneal (IP) administrations. The PAOPA was added equally to both the CMCh and the SNP solutions and allowed to sit for 24 h to allow for any polymer-drug interactions to occur before gelation. All precursor solutions were passed through a 0.45 m filter (VWR) to sterilize them prior to injection into a live animal.

To induce a model of schizophrenia, MK-801 (concentration of 0.33 mg/kg) was injected via the intraperitoneal route. All MK-801 injections were given to the animals 30 minutes before conducting any behavioral test and 30 minutes after the precursors have been administered. The MK-801 model induction lasted a duration of 2 hours.

Five experimental groups were tested at different PAOPA and gel concentrations (n=4 per experimental group): A) Drug, no gel, no MK-801 (drug-only control); B) Gels, no drug, no MK-801 (material-only control); C) No drug, no gel, MK-801 (negative behavioral control); D) Drug, no gel, MK-801 (drug-induced symptom alleviation); E) Drug, gel, MK-801 (formulation-induced symptom alleviation).

To administer the payload for the five experimental groups, a single rat was removed from its cage and placed into a closed anesthesia box with transparent sides for proper observation. Aerosolized isoflurane (with oxygen) was passed into the box until the rat was determined to be anesthetized via foot pinching reflex test. The animal was properly harnessed using a surgical cloth and held firmly by one researcher, while another researcher pipetted the SNP-CHO@CMCh and/or drug solutions directly into the nostrils of each rat at a volume of 30 μL of mixed SNP-CHO and CMCh solution into each nostril. The animal was then placed back into the anesthesia box for 1-3 minutes and the administration was repeated again (30 μL per nostril), resulting in a total of 120 μL of gel to be administered to each animal. After the last administration, each rat is placed in its own recovery chamber and observations are made with regards to breathing and nasal cleaning habits. For all experiments, the hydrogel precursor solutions are pipetted into the rat's noses 30 minutes before MK-801 injection or an hour before behavioral testing.

### In Vivo Antipsychotic Behavioral Testing

Rats were assessed for social interaction with and without MK-801 and drug/formulation administration. Animals were habituated alone in the social interaction arena, a black polyvinyl open box (100×100×40 cm) placed on black polyvinyl floor, prior to testing. On the day of testing, two unfamiliar (no prior social interaction) rats were clearly marked with a non-toxic biodegradable paint on their back and were placed in opposite corners of the social interaction arena. A ceiling-mounted video camera was located above the arena to track interactions during a 5 min tracking time with the experimenter was absent from the room, with half of the room lights remaining switched on during the testing period. Total time spent in interaction was recorded for each rat and further divided into active interaction (sniffing, following, crawling over/under, grooming, and any aggressive behavior) or passive interaction (close proximity). Recordings were analyzed by blinded observers, with the interaction times assessed by each observer averaged. No animal pairing was repeated (i.e. each rat pair represented a novel social interaction), and the arena was thoroughly cleaned out (wiped down with 75% ethanol) between each social interaction recording.

### In Vivo SNP-CHO Biodistribution

To track the transport and residence time of the gel formulation, SNP-CHO were fluorescently tagged (F—SNP—CHO) with AlexaFluor Hydrazide 657 by adding 1 mg of AlexaFluor to 700 mg of SNPs in artificial cerebrospinal fluid and stirring at room temperature for 1 h. Following, sodium cyanoborohydride (2× molar excess to the amount of aldehyde groups) was added to the solution, and the flask was allowed to stir overnight to reduce the hydrazone bond. The particles were then dialyzed against MQW (6×6 hour cycles, 3500 molecular weight cut-off membrane) and lyophilized. Following, F—SNP—CHO was re-dispersed in a 35 w/v % dispersion and gelled with 2 or 4 w/v % CMCh directly within the animal nose. At t=24 hrs (1 day) and t=72 hrs (3 days), the animals were sacrificed and the whole brain (with separate sections of the cerebellum and olfactory bulb), liver, lung, kidney, spleen, and nasal tissue were removed. The tissues were washed using saline and then frozen in an aluminum foil-wrapped container. Following, the tissue samples were homogenized in saline, and the fluorescence of the resulting mixtures was analyzed using an Infinite 200 Pro plate reader (λₑₓ=633 nm, λₑₘ=683 nm) for their relative fluorescence values. The concentration of F—SNP-CHO in each organ was then estimated based on a standard curve of F—SNP-CHO in saline suspension.

### In Vivo L-DOPA Anti-Oxidation Properties of SNP-APBA

200 μL of 25 w/v % (DS 0.5) SNP-CHO or SNP-APBA was combined with 200 μL of CMCh (pH 7) to form a 400 μL bulk hydrogel in a 12 plate well. Gels were loaded with 3 mg of L-DOPA by suspending the powered drug into the aqueous SNP precursor before gelling. The gels were submerged in 3 mL of 0.1M MES (pH 6) buffer or 10 mM PBS (pH 7.4) buffer. Oxidation of L-DOPA was assessed visually, with the formation of dark yellow-brown and black precipitates indicating L-DOPA oxidation to its dopaquinone and dopachrome polymer forms, and assessed with respect to controls of L-DOPA at the same concentration in each of the respective buffers (without a hydrogel).

For all behavioral and biodistribution tests, the data was collected and processed using a one-way analysis of variance (ANOVA) with an alpha value of 0.05 along with a Tukey's post-hoc test to determine specific groups with statistical differences.

### In Vivo Parkinsonian Model Induction to Measure Delivery of L-DOPA to the Brain

The stability of L-DOPA delivered using the SNP-CHO@CMCh or SNP-APBA@CMCh bulk hydrogels was assessed using an in vivo C. elegans assay. Wild-type late larval stage worms were exposed to 30 mM 6-OHDA in ascorbic acid for 1 hr to induce dopaminergic neurodegeneration. After 24 hours, worms were added to a 12-well plate in which each well contains 400 μL of SNP-CHO@CMCh or SNP-APBA@CMCh bulk hydrogel as well as E. coli (for food) to form a 3 mL liquid culture in which worms are continuously exposed to released L-DOPA.

### In Vivo Parkinsonian Movement Testing

Worms were sampled at various time points (0 h, 4 h, 8 h, 12 h, 24 h, 48 h) to assess movement. Motility changes, assessed based on the average speed of the worms in mm/s (minimum n=20 worms per sample and time point tested), were measured using an in-house voltage-induced electrotaxis microfluidic assay developed to quantify drug induced dopaminergic neuron signal-mediated movement. In addition to the test sample (Group 1), control groups of no 6-OHDA, no hydrogel (Group 2, untreated control), 6-OHDA, no hydrogel (Group 3, drug control); 6-OHDA, blank hydrogel (Group 4, material control); and 6-OHDA, drug(s) without hydrogel (Group 5, delivery vehicle control) were assessed (minimum n=20 worms/group). L-DOPA was loaded in both SNP-CHO@CMCh and SNP-APBA@CMCh 25 w/v % hydrogels (DS=0.5) at 3 or 9 mg/400 mL hydrogel.

### Results and Discussion

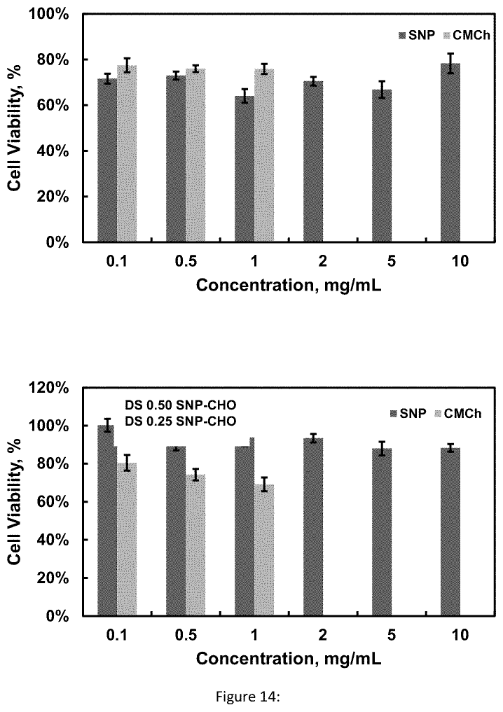

The cytotoxicity of CMCh and various functionalized SNP-CHO gel precursor materials to SH-SY5Y neuroblastoma HNEpC primary human nasal epithelial cells is shown in FIG. 14. The 0.25 mol SNP-CHO samples showed high cell viability (>80%) at all concentrations tested (0.1-10 mg/mL), while the 0.50 mol SNP-CHO samples showed no cytotoxicity at concentrations from 0.1-1 mg/mL but lower viabilities (<70%) for concentrations between 2-10 mg/mL; note that these concentrations are anticipated to be significantly higher than the weight percentages seen in vivo given the degradation kinetics previously described. The cytocompatibility of CMCh similarly was tested for concentrations 0.1-1 mg/mL, maintaining the same SNP/CMCh ratio used in the lead formulations; similarly high >80% cytocompatibility values were maintained with CMCh.

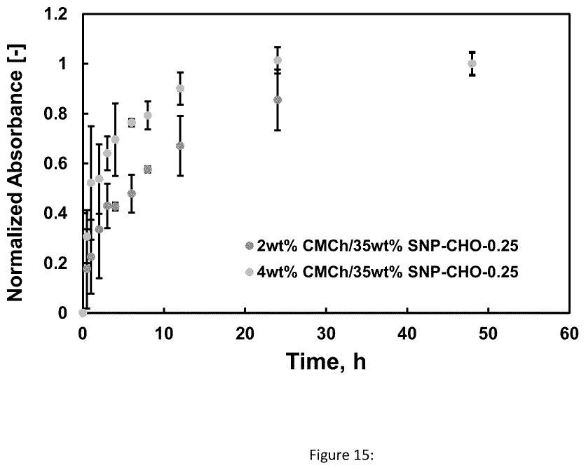

In vitro release studies were subsequently conducted to assess the relative release rates from PAOPA-loaded hydrogels (FIG. 15). Since the HPLC signals of both SNPs and PAOPA overlapped over a range of elution solvent concentrations chromatogram signals were normalized based on their (plateau) equilibrium 48 h maximum cumulative absorbance values to enable comparisons between the different hydrogels. The less crosslinked 2 wt % CMCh/35 wt % SNP-CHO-0.25 hydrogels released PAOPA slower than the more crosslinked and denser 4 wt % CMCh/35 wt % SNP-CHO-0.25 hydrogel, a result we attribute to: (1) the significantly higher swelling of the 4 wt % CMCh hydrogel increasing diffusion-based release of PAOPA from the gel; and (2) the increased occupancy of the available aldehyde functional groups on SNP-CHO-0.25 with the higher number of amine groups in the 4 wt % CMCh hydrogel, weakening the Schiff base hydrogel/PAOPA interactions to promote faster drug release.

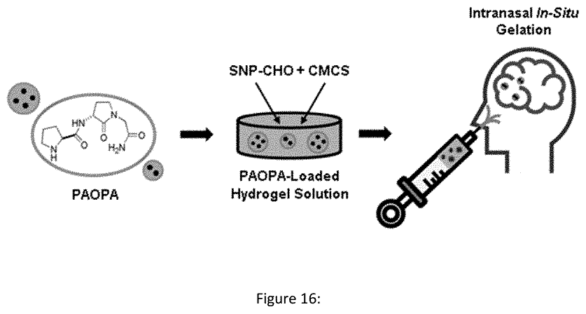

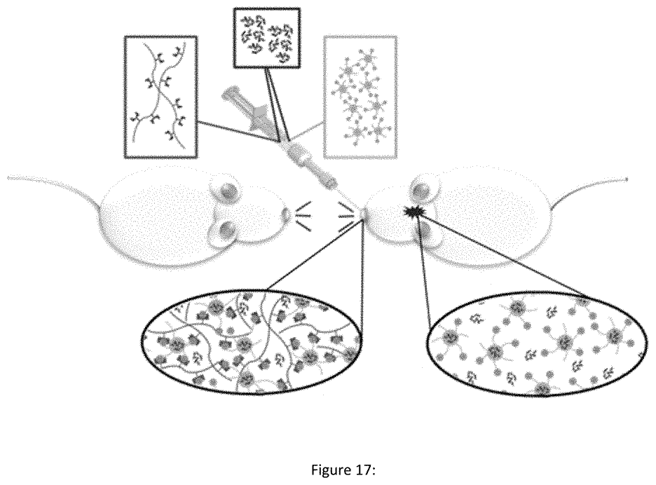

The method of administering the gels intranasally is shown schematically in FIG. 16, while the changes in total social interaction time for various treatment groups are shown in FIG. 17. In the absence of MK-801 knockdown (panel A), groups treated via IN of PAOPA only (0.5 mg/kg) or unloaded hydrogel formulations (2 w/v % CMCh/35 w/v % SNP-CHO-0.25 or 4 w/v % CMCh/35 w/v % SNP-CHO-0.25) showed neither increased nor decreased total interaction times relative to the negative control rat that received no treatment. The PAOPA result is consistent with the literature, in which delivery of PAOPA at 1 mg/kg via IP did not affect social interaction time. When MK-801 was administered (panel B), injection with aCSF only resulted in significant decreases in total interaction times (F(5,16)=41.782, p<0.00001; post hoc, *p<0.01). Administration of PAOPA (0.5 mg/kg) at an acute time point 30 minutes before MK-801 challenge demonstrated the expected attenuation of social interaction deficits when assessed 30 minutes post-MK-801 administration, however, when groups treated with PAOPA were challenged with MK-801 24 hours later without further drug administration, the benefits of PAOPA were no longer observed (F(5,16)=41.782, p<0.00001; post hoc, *p<0.01 comparing 0 hr and 24 hr PAOPA-only treatments), indicating rapid clearance of PAOPA and mandating repeated administrations of PAOPA for any observed clinical benefit. In contrast, when the same 0.5 mg/kg dose of PAOPA was delivered in the 2 w/v % CMCh/35 w/v % SNP-CHO-0.25 hydrogel (panel C), full symptom alleviation relative to the negative controls (panel A) was observed for as long as 3 days post-administration (F(5,19)=28.23, p<0.00001; post hoc, *p<0.01), with significant increases in social interaction time still observed as long as five days post-administration. The 4 w/v % CMCh/35 w/v % SNP-CHO-0.25 hydrogel (panel D) exhibits similar positive effects relative to the positive aCSF control over the same 5-day period but only achieves comparable social interaction times to negative control results at the 24 hour time point, with an apparent induction time at shorter time points and a tailing off of drug efficacy at longer time points. Without wishing to be bound by theory, the higher viscosity and longer degradation time of the 4 w/v % CMCh formulation results in more patchy deposition upon injection and slower PAOPA release, reducing its efficacy relative to the 2 w/v % CMCh formulation. Over the course of 7 days (168 hours), both groups presented social interaction times comparable to the aCSF controls, indicating either full degradation of gels or depletion of PAOPA in the gels to a sub-clinical dose.

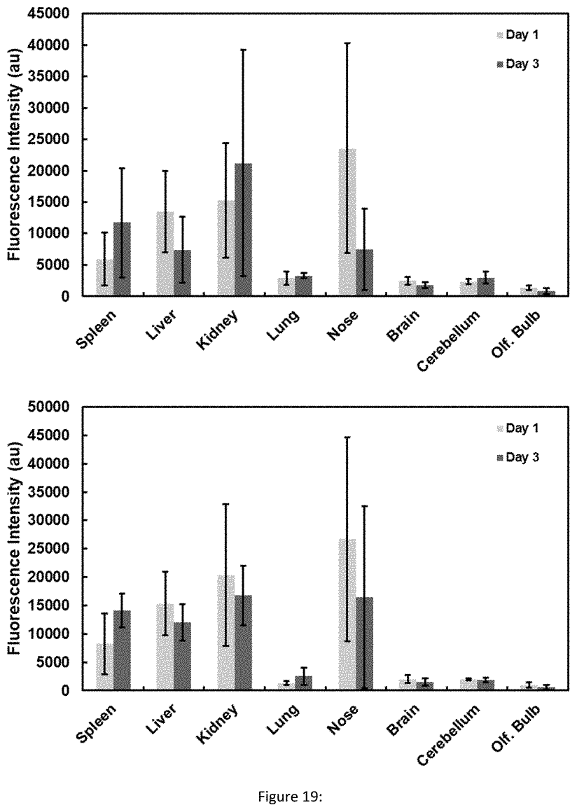

The biodistribution of released SNP-CHO following gel degradation was assessed by fluorescently labeling the starch nanoparticles and grinding the organs recovered at specific timepoints following sacrifice. On day 1, for both the 2 w/v % and 4 w/v % CMCh/35 w/v % F—SNP-CHO-0.25 gels, the largest fluorescence intensity was detected in the nose, with significant but lower fractions detected in typical nanoparticle clearance organs (e.g. spleen, liver, kidney) and very low fractions present in the lung (FIG. 19). This result suggests good retention of the administered formulation in the nasal cavity, accounting for the effective longer-term behavioral benefits observed with the gel delivery vehicles. Note that the larger error bars associated with the nose readings are associated with the very small volume of the nasal cavity collected and thus more animal-to-animal sampling errors in isolating only the nasal cavity tissue. While fluorescence in the different collected brain regions was lower than that observed in clearance organs, substantial signals related to the presence of the SNPs throughout the brain are still measured that clearly demonstrate transport of SNPs to the brain. On day 3, similar overall patterns in biodistribution were observed, albeit with lower concentrations in the nose, corresponding similar or slightly lower (but still significant) concentrations in most brain regions, and slightly higher concentrations in clearance organs. Thus, the in situ gelling hydrogel is demonstrated to be both effectively retained in the nose over several days and enable the transport of the SNP component at least into the brain, as desired for effective drug delivery.

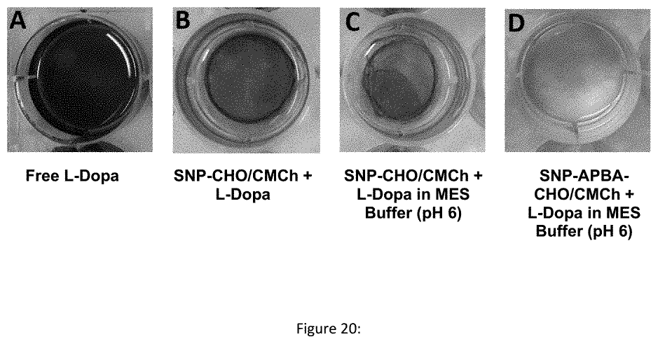

In another embodiment, levodopa (L-DOPA) was used as the target drug to measure the efficacy of the SNP-CHO@CMCh and SNP-APBA@CMCh materials as drug carriers for the intranasal delivery of dopamine precursors and agonists. L-DOPA oxidizes quickly at physiological pH, as indicated by a color change from clear to yellow, orange, and ultimately black as it transitions between its dopaquinone, dopachrome and melanin oligomer forms. The boronate ester group on APBA can bind via a reversible covalent bond with the vicinal diol group on L-DOPA, an interaction hypothesized to slow L-DOPA oxidation. Wells were inspected after 12 h of incubation at 37° C. Free drug in both buffers tested became black with oxidation (FIG. 20-A). In contrast, SNP-CHO-based hydrogels become yellow-brown indicative of partial drug oxidation (FIG. 20-B/C) while hydrogels prepared with SNP-APBA (FIG. 20-D) hydrogels had minimum discoloration. As such, while all gel formulations tested significantly impaired L-DOPA oxidation, the ABPA-functionalized hydrogels could almost fully suppress L-DOPA oxidation over the tested 12 h period, a result attributed to the capacity of the phenylboronic acid group in SNP-APBA to form a reversible covalent bond with the cis-diol group in L-DOPA.

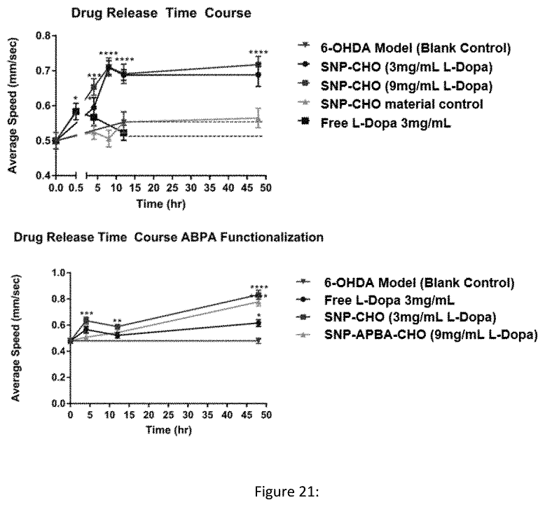

L-DOPA hydrogels facilitated drug release to the worms that allowed for improved swimming speed relative the control 6-OHDA model for 48 hours, while the free drug only provided benefit for 4 hours (FIG. 21). Worms without the hydrogel did not survive the full 48 h, possibly due to lack of mobility or stress induced from L-DOPA oxidation that is avoided in the presence of the hydrogel (due to both controlled release of L-DOPA and the role of the hydrogel in suppressing L-DOPA oxidation). Both SNP-CHO@CMCh and SNP-APBA@CMCh were highly effective at restoring worm motility over the entire 48 h observation period. The SNP-APBA@CMCh gel shows a reduced effect over the first 12 h compared to that prepared with the unfunctionalized nanoparticles, suggesting slower L-DOPA release from these gels as anticipated due to the cis-diol/boronic acid covalent interaction but still an extended positive therapeutic effect over at least 48 hours.

### Example B1: Synthesis of Starch Nanoparticles and Functionalized Chondroitin Sulfate Hydrogel Prepolymers

### Materials

Chondroitin sulfate A sodium salt from bovine trachea (CS, average molecular weight=25 kDa, Millipore Sigma), 1′-ethyl-3-(3-dimethylaminopropyl)-carbodiimide (EDC, Carbosynth, commercial grade), cystamine dihydrochloride (CysHCl, Millipore Sigma, 96%), dithiothreitol (DTT, 1M in H₂O, BioUltra), sodium periodate (NaIO₄, Millipore Sigma, 99%), L-glutathione oxidized (GSSG, Millipore Sigma, 98%), L-glutathione reduced (GSH, Millipore Sigma, 98%), Span® 80 (Millipore Sigma), polyglycerol polyricinoleate 4175 (PGPR, Palsgaard), fractionated coconut oil 100% MCT oil (Voyageur Soap & Candle Co.), reagent grade acetone (Caledon Laboratories), ethylenediaminetetraacetic acid (EDTA, Millipore Sigma, 99%), 5,5′-dithio-bis-(2-nitrobenzoic acid) (DTNB, Millipore Sigma, >98%), sodium cyanoborohydride solution (NaCNBH3, 5 M solution in 1 M NaOH, Millipore Sigma), sodium phosphate dibasic (Na2HPO4, Millipore Sigma), doxorubicin hydrochloride (DOX, Millipore Sigma, pharmaceutical secondary standard) and resazurin sodium salt (Millipore Sigma) were all used as received. Phosphate buffered saline solution was made from tablets (PBS; contains 10 mM phosphate buffer, 140 mM NaCl, 3 mM KCl, and pH 7.4 when dissolved in 200 mL water, Millipore Sigma). SNPs (EcoSynthetix, experimental grade) were dialyzed against DIW for a minimum of 6 (6+ hour) cycles before use. For all experiments, Milli-Q water was used. B16-F10 mouse skin melanoma cells, CT26 colon carcinoma cells, and NIH 3T3 mouse fibroblasts were all obtained from a collaborator. Media contents including Dulbecco's Modified Eagle Medium-high glucose (DMEM), fetal bovine serum (FBS), penicillin streptomycin (PS), and trypsin-EDTA and were purchased from Invitrogen Canada (Burlington, ON).

### Synthesis of Thiol Functionalized Chondroitin Sulfate (CS-SH)

CS (4.0 g, 0.16 mmol, 54 monomeric equivalents) was placed in 125 mL of water and bath sonicated for 1 hr to dissolve. Then, CysHCl (2.62 g, 17.3 mmol, 2 eq of CS) and EDC (2.68 g, 17.3 mmol, 2 eq of CS) were added to CS and the pH was adjusted to 4.75. The pH was subsequently maintained between 4.5 and 5 with dropwise 0.1 M HCl for 6 hrs. The resulting solution was dialyzed against H₂O for a minimum of 6 (6+ hr) cycles in 10 kDa MWCO dialysis tubing. Subsequently, the purified polymer was removed from dialysis, the pH was adjusted between 8 and 8.5 with 0.1 M NaOH solution, and DTT (3.2 mL, 21.6 mmol, 2.5 eq of CS) was added and reacted for 6 hrs between pH 8 and 8.5. Upon completion, the reaction was adjusted to pH 3.5 to prevent disulfide re-crosslinking. Dialysis at pH 3.5 was done for 4 (6+ hr) cycles followed by lyophilization to yield purified CS-SH. The degree of substitution of CysHCl was quantified using conductometric base-into-acid titration, while the degree of substitution (DS) of free thiols was determined through an Ellman's assay performed following manufacturer's instructions.

### Synthesis and Characterization of Aldehyde-Functionalized SNPs (SNP-CHO)

Methods for SNP-CHO synthesis and characterization were completed as described in Example A1.

### Synthesis of Thiol-Functionalized SNPs (SNP-SH)

SNP-CHO (4 g) was dispersed in 75 mL of H₂O at 45° C. under 200 rpm magnetic stirring. The pH was adjusted to 5 and, subsequently, CysHCl (4.09 g, 2 eq of SNP-CHO) was added. The mixture was reacted under stirring for 4 hrs. Then, NaCNBH₃ solution (0.75 mL, 2 eq of SNP-CHO) was added and the pH was corrected to 8.5 before reacting for another 4 hours at room temperature. The aggregated product was dialyzed for 6 (6+ hr) cycles. DTT (3.38 mL, 21.6 mmol, 2.5 eq of SNP-CHO) was then added and reacted for 6 hrs at 45° C. Finally, the pH was corrected to 3.5 to prevent disulfide re-crosslinking. Dialysis and thiol quantification were completed as described above for CS-SH.

### Results and Discussion

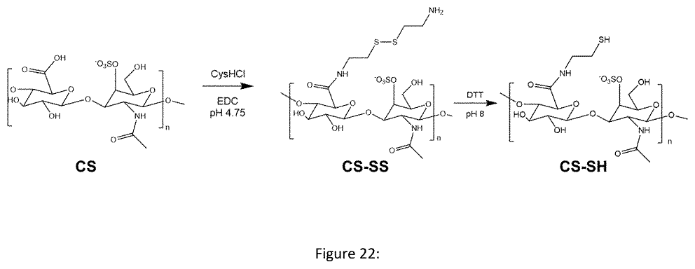

CS was chemically functionalized with thiol groups to enable crosslinking, while SNPs were functionalized with aldehydes to improve drug loading with amine-bearing chemotherapeutic drugs such as doxorubicin hydrochloride (FIG. 22). To prevent unwanted aggregation and increase the control of nanogel formation, disulfide bonds were reduced through a disulfide exchange with DTT followed by intramolecular exchange to yield CS-SH. The DS for CS-SS was 0.37, as quantified by base-into-acid titration, while the DS for CS-SH was 0.17, as quantified by the colorimetric Ellman's assay; this DS enables rapid gelation in the presence of GSSG (Table 1).

SNP-CHO was quantified via titration after reacting with hydroxylamine, yielding an aldehyde DS of 0.32. The porous nature of SNPs already allows the physical entrapment of a drug⁴⁰; the presence of aldehydes may increase the loading of amine-bearing anti-cancer drugs (such as model drug doxorubicin) through imine bonds.

### Example B2: Synthesis and Physiochemical Characterization of Starch Nanoparticles and Functionalized Chondroitin Sulfate Hydrogels and Nanocluster Nanogels

### Synthesis of Starch Nanoparticle and Chondroitin Bulk Gels (CS-SH@SNP-CHO)

Bulk gels were made with varying concentrations of precursors to assess the gelation time prior to nanocluster nanogel formation. All gels were made with a total volume of 1 mL, where the volume ratio of CS-SH to SNP (and its derivatives, SNP—CHO and SNP-SH) was always 1:1. 0.5 mL of each of the CS-SH and SNP-CHO precursor solutions were added to a 3 mL glass scintillation vial and vortexed lightly for 1 minute. Following, 0.02 mL of a 50 mM GSSG stock solution was added to the precursor mixture and vortexed lightly for 30 seconds. Gel precursors were left on the benchtop at room temperature for 6 hours to enable gelation. Vial inversion tests were done to quantitatively determine gelation time, with samples deemed to be “fully gelled” when inverting the vials yielded no flow after 5 seconds. Redox responsiveness of these gels was tested by adding 5 molar equivalents of DTT to vials followed by vortexing, with de-gelling kinetics assessed by the vial inversion method 90 minutes after DTT addition. Synthesis of Starch Nanoparticle and Chondroitin Nanocluster Nanogels (CS-SH@SNP-CHO)

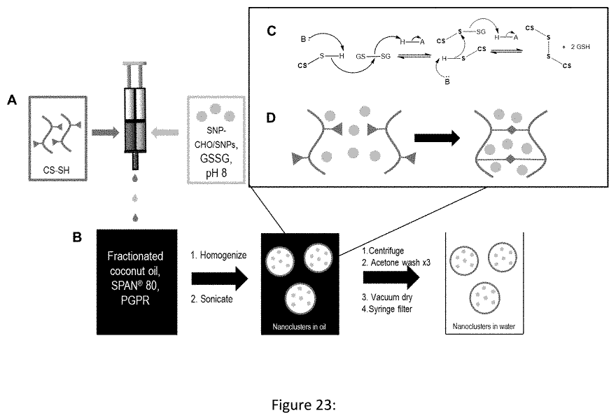

The method used to fabricate nanocluster nanogels is shown schematically in FIG. 23. A 20 w/v % solution of CS in H₂O, 5 w/v % solution of SNP-CHO and 100 mM solution of GSSG in Ellman's buffer (0.1M Na₂HPO₄, 1 mM EDTA, pH 8) were bath sonicated with heat for 30 minutes to fully disperse. In parallel, a 50 mL Falcon tube containing fractionated coconut oil (25 mL, HLB value=4 to 5), Span® 80 (2 mL, HLB=4.3), and PGPR (0.5 mL, HLB 1.5-2) was homogenized at 12,000 rpm for 5 seconds to create the oil phase. CS-SH (0.25 mL, final concentration=10 w/v %) was then loaded into one barrel of a double-barreled syringe while SNP-CHO (0.2 mL, final concentration=2 w/v %) and GSSG (0.05 mL, final concentration=10 mM) were loaded into the other barrel (FIG. 23-A). Precursor solutions were dispensed through the mixing chamber of the double-barreled syringe and into the emulsifying mixture of fractionated coconut oil containing two emulsifiers, Span© 80 (HLB=4.3) and PGPR (HLB=1.5 to 2) (FIG. 23-B). The emulsion was homogenized using a T25 Digital Ultra Turrax homogenizer (IKA, Staufen, Germany) for 15 minutes at 12,000 rpm followed by probe sonication (Q700a sonicating probe, QSonica, Newtown, Conn., USA, total energy between 10,400-10,500 J, ˜3 min.), all on an ice bath to reduce the size of the emulsified droplets while disulfide exchange-mediated gelation occurred (FIG. 23-C). The CS-SH@SNP-CHO nanocluster nanogel product was immediately transferred to a scintillation vial and cooled to room temperature on benchtop (about 3 to 4 hrs) before purification.

### Purification of CS-SH@SNP-CHO Nanocluster Nanogels

CS-SH@SNP-CHO nanocluster nanogels were purified by consecutive centrifugation and acetone wash steps. Briefly, CS-SH@SNP-CHO (3 mL) nanocluster nanogels were transferred to 15 mL Falcon tubes and centrifuged at 10,400×g for 20 minutes with the supernatant discarded. Nanocluster nanogels were washed with acetone (2 mL) before repeating centrifugation. The acetone supernatant was discarded, and the nanocluster nanogels were washed with acetone (1 mL) and centrifuged again. The supernatant was again discarded, and the nanocluster nanogels were placed in a vacuum oven at 55° C. for 30 mins, yielding fully purified CS-SH@SNP-CHO nanocluster nanogels.

### Characterization

### Dynamic Light Scattering (DLS)

CS-SH@SNP-CHO nanocluster nanogels were dispersed in H₂O with a concentration of 2 mg/mL and vortexed (1600 rpm, setting 5) for 1 minute. Vortexed samples were filtered with a 0.45 m PTFE syringe filter to remove any large aggregates. Filtered nanoclusters were added to a clear polypropylene cuvette and measured on a NanoBrook 90Plus instrument (Brookhaven, Long Island, N.Y., USA; temperature=25° C.).

### Zeta Potential and Electrophoretic Mobility

CS-SH@SNP-CHO nanocluster nanogels were dispersed in a 10 mM NaCl solution at a concentration of 2 mg/mL and vortexed and filtered as with DLS. Filtered nanocluster nanogels were added to a clear polypropylene cuvette and measured on a NanoBrook 90Plus instrument operating in phase analysis light scattering (PALS) mode (Brookhaven, Long Island, N.Y., USA; temperature=25° C.).

Size Distribution with Nanoparticle Tracking Analysis (NTA)

Nanocluster nanogels prepared for DLS were further diluted 10× in water (final concentration=0.2 mg/mL). 0.5 mL of the suspension was added directly to the microscope stage, while a syringe pump was used to flow the remaining nanocluster nanogels across the stage for the measurement. Single nanoparticle tracking analysis (NTA 3.4) was analyzed with a LM14 HS NanoSight microscope (Malvern Panalytical, Worcester, UK; 100 to 200 particles per frame).

### Transmission Electron Microscopy (TEM)

Transmission electron microscopy (TEM) was performed using a JEOL 1200EX TEMSCAM instrument to access nanocluster size and structure. Samples were prepared as described in Section A1.

### Results and Discussion

Prior to nanocluster nanogel formation, the gelation kinetics of CS-SH@SNP-CHO bulk gels were assessed with vial inversion tests to determine the concentrations necessary for rapid gelation (Table 1). SNP-SH hindered gel formation, as indicated by the increased gelation time necessary for CS@SNP-SH gels as the SNP-SH concentration increased and the lack of gel formation in SNP-SH-only gels. Increasing the CS-SH concentration decreased gelation time in 11 of 15 compositions where CS-SH was varied, suggesting that increased disulfide formation decreases gelation time. GSSG significantly accelerates disulfide-crosslinked CS gel formation, as only 1 mM GSSG induced gelling 3.2 times faster in 15 w/v % CS-SH gels relative to a 30 w/v % CS-SH precursor solution without GSSG. Further, increasing the concentrations of unfunctionalized SNPs and SNP-CHO decreased the gelation time, with 1 w/v % SNP-CHO showing considerably shorter gelation times (˜10 minutes) compared to 1 w/v % SNPs (˜80 minutes). An optimum ratio of CS-SH:SNP-CHO:GSSG was determined to be 10:2:10 to enable nanogel formation in under 20 minutes.

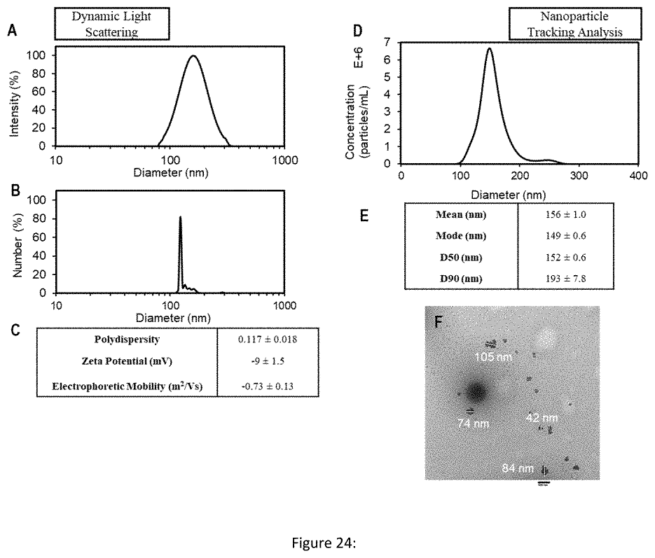

The size and surface properties of CS-SH@SNP-CHO nanocluster nanogels were analyzed to ensure that the size of the nanoclusters was between 100 and 200 nm (for prolonged circulation and effective tumour uptake), and CS-SH was encapsulating the SNP-CHO nanoparticles (FIG. 24). Both intensity (FIG. 24-A) and number (FIG. 24-B) size distributions indicate relatively monodisperse (polydispersity <0.2) single particle populations with average diameters between 100 and 200 nm, suggesting that nanogels are formed and that there are few free-floating SNPs in suspension. NTA showed similar size distributions (average particle size ˜150 nm, FIG. 24-D) and narrow polydispersities (D90 values <200 nm, FIG. 24-E), confirming that monodisperse nanocluster nanogels in the desired size range are formed. Finally, electrophoretic mobility measurements show the nanocluster nanogels are anionic (−0.73 m²/Vs), suggesting that some of the negative charges on CS are present on the surface of CS-SH@SNP-CHO nanoclusters (FIG. 24-C). The morphology of nanoclusters as shown by TEM suggest that SNP-CHO are adequately clustered due to the emulsion process (FIG. 24-F). Without distinct phase separation between the SNPs and CS-SH, it is suggested that disulfide crosslinked CS-SH is entangled between SNPs rather than as a distinct “shell”. Clusters show a slightly smaller size of ˜40-100 nm on TEM compared to DLS and NTA, which is a common drying artefact of nanogels on TEM. Densely packed, oblong CS-SH@SNP-CHO nanoclusters differ from the spherical morphology of 20 nm SNP-CHO (FIG. 3), suggesting that multiple SNPs were clustered together.

### Example B3: Biological Applications of Starch Nanoparticle—Chondroitin Sulfate Nanocluster Nanogels

Doxorubicin Hydrochloride (DOX) Loading into Nanoclusters

DOX was loaded into nanoclusters by mixing followed by purifying through centrifugation. First, a 15 mg/mL DOX standard in either H₂O or DMSO was created by sonicating under minimal heating (˜40° C.) for 15 mins. 4 mg of purified nanoclusters were dispersed in 2 mL H₂O in a 7 mL glass scintillation vial in parallel with the DOX standard. Various initial concentrations of each DOX standard—0.1 w/v % (2 μL), 0.5 w/v % (10 μL), 1% (20 μL), 5% (100 μL), and 10% (200 μL)—were added to purified clusters and mixed with a magnetic stir bar for 24 hrs. DOX-loaded CS—SH@SNP-CHO nanoclusters (DOX—CS—SH@SNP—CHO) were centrifuged at 10,400×g for 20 minutes, and the fluorescence of the supernatant was measured using a BioTek Synergy HTX Multi-Mode Reader plate reader (λₑₓ: 480+/−15 nm, λₑₘ: 595+/−15 nm). The loading capacity (LC) and encapsulation efficiency (EE) were quantified by comparing to a calibration curve of DOX in its respective solvent (water or DMSO). All loading experiments were done in triplicate.

### Nanocluster Breakdown In Vitro GSH Solutions

Purified CS-SH@SNP-CHO nanoclusters were resuspended in 10 mM PBS to a concentration of 1.5 mg/mL. Samples were vortexed for 1 min at 1600 rpm, bath sonicated for 10 minutes, and then syringe filtered as described above. Particle size was measured using dynamic light scattering every 90 s. After 3 measurements, a volume of a 100 mM GSH stock solution in PBS (15 mg in 0.5 mL) was added to the cuvette to reach different target concentrations of GSH and final volume of 2.75 mL. Effective diameter measurements were taken every 3 minutes for the first hour then once every hour over the next 12 hours. All experiments were done in triplicate.

DOX Release from Nanoclusters Via Membrane Separation

DOX release was measured in various concentrations of GSH via membrane separation. First, 0.5 v/v % Tween 80 was added to 10 mM, pH 7.4 PBS buffer to increase the solubility and transport of DOX through the membrane as well as to simulate endosomal transport through biological barriers. Then, varying concentrations of GSH were added to simulate GSH concentrations in normal and disease states, both intracellularly and extracellularly. DOX-nanoclusters were dispersed to a final concentration of 0.2 mg/mL in the desired release buffer, mixed well, and bath sonicated for several minutes to properly disperse. 1 mL of DOX—CS—SH@SNP-CHO were added to cellulose acetate Float-a-Lyzers, placed in a 50 mL Falcon tube containing 20 mL of release media, and shaken slowly in an incubator at 37° C. and 150 rpm. 1 mL of media outside of the dialysis tubing was collected and replaced with fresh media after 0, 1, 2, 4, 8, 24, 48, 72, 96, 120, 144, and 168 hours after incubation. Fluorescence was measured using a BioTek Synergy HTX Multi-Mode Reader microplate reader (λₑₓ: 480+/−15 nm, λₑₘ: 595+/−15 nm), with the corresponding drug release calculated relative to a DOX calibration curve. Release was repeated with DOX-only controls, wherein an equivalent concentration of free DOX in PBS (no GSH) was added to Float-a-Lyzers and released over time. All release experiments were done in triplicate.

### In Vitro Cell Viability Assay

The cytotoxicity of the fabricated nanoclusters towards B16-F10 melanoma cells, CT26 colon carcinoma, and NIH 3T3 fibroblasts (non-cancerous, control cells) with or without DOX loading was assayed in 96 well plates. 200 μL of the cell suspension (2.5×10⁴ cells/mL) in DMEM medium was seeded into the wells of a 96 well plate. One day after the cell attachment, blank nanoclusters (control), free DOX, and DOX—CS-SH@SNP-CHO of various DOX concentrations (0, 0.001, 0.01, 0.1, 1.0 and 10.0 μg/mL) were added into separate wells. After 24 hrs and 72 hrs of incubation, the media was removed and resazurin solutions pre-dissolved in media were added into the wells as per the manufacturer's instructions. After 4 h of incubation, the fluorescence intensity was measured using a Tecan Infinite M200 Pro plate reader using an excitation wavelength of 560 nm and an emission wavelength of 590 nm. All measurements were done in triplicate, with the error bars representing the standard deviation of the replicate measurements.

### Results and Discussion

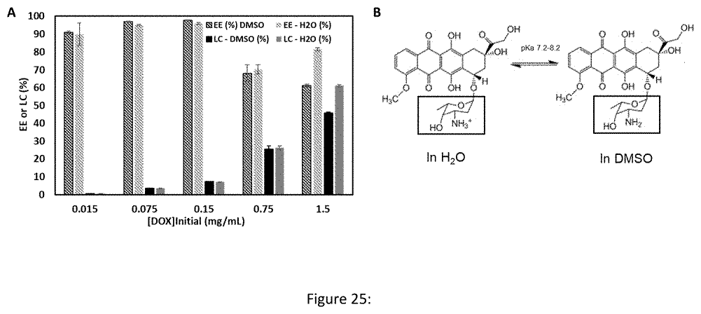

DOX was loaded into CS-SH@SNP-CHO nanoclusters under various conditions to maximize the amount of DOX loading into clusters. The EE and LC were calculated by comparing to a calibration curve in either water or DMSO (FIG. 25). EEs—a measure of the amount of starting DOX was efficiently encapsulated in clusters—exceed 90%; however, LCs—a measure of how much DOX is bound per mass of nanocluster—are less than 10% when initial DOX concentrations are at or below 0.15 mg/mL (FIG. 25-A), indicating that at low DOX concentrations many binding sites are still available on the nanoclusters. Above 0.75 mg/mL, more available DOX binding sites can be used and the LCs increase to above 25%. Above 1.5 mg/mL DOX, the EE and LC are significantly greater when DOX is in water rather than DMSO, likely due to the protonation of the amine on DOX when in water that is not present when dissolved in DMSO (FIG. 25-B). Protonated DOX is available to bind to negatively-charged CS through electrostatic interactions while also forming imines with free aldehydes on SNPs. This dual-loading mechanism in water yields greater loading potential than in DMSO that only allows imine formation.

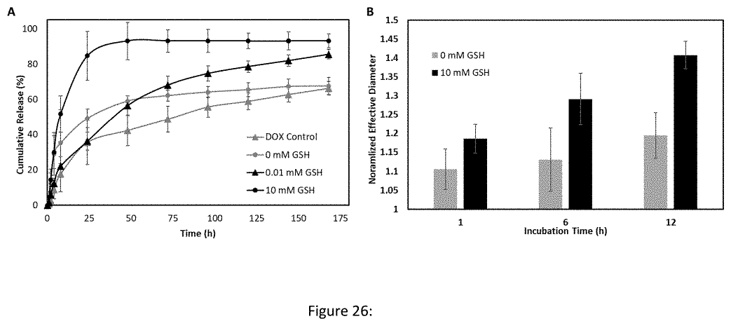

The ability of nanoclusters to release anti-cancer therapeutics when subjected to cancer-like microenvironments was tested by incubating DOX—CS-SH@SNP-CHO in media rich in glutathione (GSH), a disulfide-reducing tripeptide that is commonly overexpressed in numerous cancer cell types that could reduce the disulfide bonds between CS-SH to release SNP-bound DOX. Release of DOX from nanoclusters incubated in either 0, 0.01, or 10 mM of GSH for 7 days was quantified via fluorescence (FIG. 26-A). A GSH- and time-dependent release was noted, with the incubation of the nanoclusters in 10 mM GSH resulting in the release of more total faster; more specifically, after 24 h, more than 84% of DOX was released, which was significantly higher than that achieved in the presence of 0 mM (49%) or 0.01 mM (36%) GSH and confirms the key role of the reducing agent in driving microenvironment-specific release. The enhanced release of CS-SH@SNP-CHO nanoclusters in GSH may be explained by the increased swelling of nanoclusters as dilsulfide bonds are de-crosslinked at 10 mM GSH, as supported by increasing effective diameters when GSH-incubated clusters were measured via DLS (FIG. 26-B). Higher concentrations of GSH also show greater overall release after 7 days, as 10 mM GSH instigates a release of over 93% while 0 mM and 0.01 mM GSH yield 67% and 85% release, respectively.

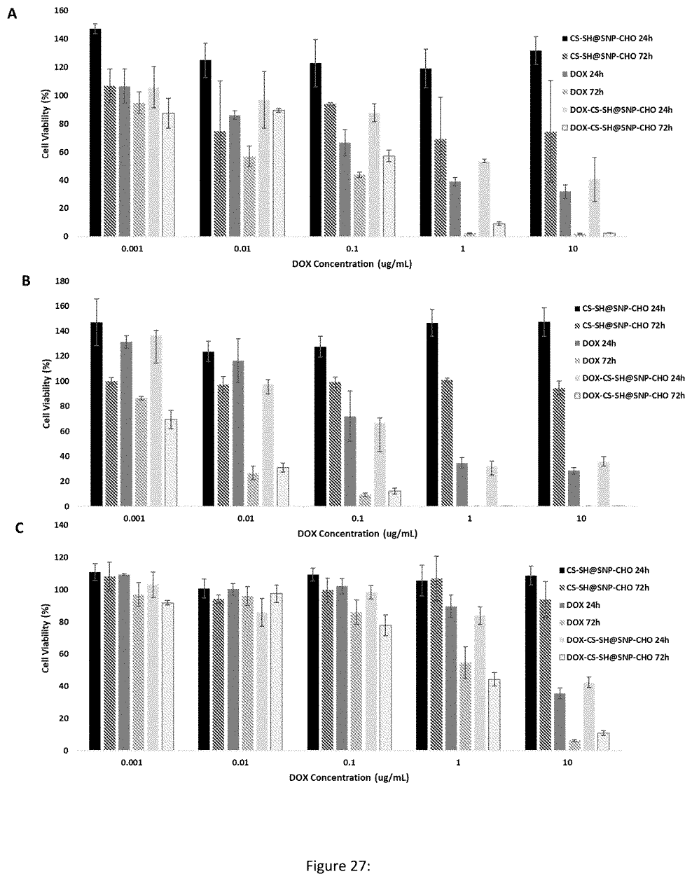

To test the practical cancer-killing potential of nanoclusters, 3 different cell lines were cultured in vitro and the viabilities of these cell lines were examined after incubating with varying concentrations of DOX either in or out of clusters over 24 and 72 hours. NIH 3T3 fibroblasts were used as control (non-cancerous) cells while two cancerous cell lines—B16-F10 melanoma and CT26 colon carcinoma—were compared to see if the cancer-killing potential could be maintained in cells with differing cell architectures. Blank CS-SH@SNP-CHO nanoclusters were not cytotoxic, showing viability >80% after 72 h in all cell lines. Cell viability decreased in a time- and concentration-dependent manner when incubated with DOX—CS-SH@SNP-CHO up to 10 μg/mL in all cell lines. In non-cancer 3T3 cells, there was significantly less cell toxicity after both 24 and 72 hours when cells were incubated with DOX—CS-SH@SNP-CHO relative to free DOX (FIG. 27-A), consistent with the entrapment of at least a portion of the DOX in the nanoclusters over the course of the experiment. Both cancer cell lines with ≥1 μg/mL DOX showed no statistical difference in anti-cancer ability between free DOX and DOX—CS-SH@SNP-CHO (FIG. 27-B,C), suggesting that DOX—CS-SH@SNP-CHO can utilize cancer-specific receptors and peptides to deliver DOX as quickly and effectively as free DOX in 2D cell cultures.

### Example C1: Synthesis of Aldehyde-Functionalized Cationic Starch Nanoparticles and Hydrogel Copolymer Precursors

### Materials

Poly(ethylene glycol) methyl ether methacrylate (OEGMAm, Millipore Sigma), 2,2-azobisisobutryic acid dimethyl ester (AIBMe, Wako Chemicals, 98.5%), acrylic acid (AA, Millipore Sigma, 99%), thioglycolic acid (TGA, Millipore Sigma, 98%), 1,4-dioxane (Caledon, 99%), adipic acid dihydrazyde (ADH, Alfa Aesar, 98%), 1′-ethyl-3-(3-dimethylaminopropyl)-carbodiimide (EDC, Carbosynth, commercial grade), 2,3-dimethylmaleic anhydride (DMA, Millipore Sigma, 98%), hydrochloric acid (HCl, 1 M, LabChem), sodium hydroxide (NaOH, 1 M, LabChem), 4-bromobenzaldehyde (Millipore Sigma, 99%), N,N-dimethylformamide (DMF, Millipore Sigma, 99.8%), doxorubicin hydrochloride (DOX, Millipore Sigma, pharmaceutical secondary standard) and resazurin sodium salt (Millipore Sigma) were all used as received. B16-F10 mouse skin melanoma cells were obtained from collaborator. Media contents including Dulbecco's Modified Eagle Medium-high glucose (DMEM), fetal bovine serum (FBS), penicillin streptomycin (PS), and trypsin-EDTA and were purchased from Invitrogen Canada (Burlington, ON). Cationic SNPs (EcoSynthetix, experimental grade) were dialyzed against DIW for a minimum of 6 (6+ hour) cycles before use. Phosphate buffered saline solution was made from tablets (PBS; contains 10 mM phosphate buffer, 140 mM NaCl, 3 mM KCl, and pH 7.4 when dissolved in 200 mL water, Millipore Sigma). For all experiments, Milli-Q grade water was used.

C57BL/6 mice were purchased from Charles River Canada, (St. Constant, QC, Canada) and housed in a controlled environment in the Central Animal Facility at McMaster University, with food and water provided ad libitum

### Synthesis of Acid-Functionalized Poly[Oligo(Ethylene Glycol) Methyl Ether Methacrylate] (POEGMA-AA)

POEGMA-AA was prepared by adding AIBMe (115 mg, 0.499 mmol), AA (1.517 g, 21.05 mmol), OEGMA₄₇₅ (10.00 g, 21.05 mmol), and TGA (2.5 mg, 0.027 mmol) to a 250 mL single neck flask. 40 mL of dioxane was added, and the solution was purged with nitrogen for 40 minutes. The flask was sealed and submerged in a pre-heated oil bath at 75° C. for 4 hours under magnetic stirring. After polymerization, the solvent was removed by rotary evaporation at 45° C., and the POEGMA-AA copolymer was purified by dialysis against DIW for a minimum of 6 (6+ hour) cycles and lyophilized to dryness. The degree of functionalization was determined using conductometric base-into-acid titration. The polymers were stored as 20 w/w % solutions in DIW with pH 7.4 at 4° C.

### Synthesis of Hydrazide-Functionalized Poly[Oligo(Ethylene Glycol) Methyl Ether Methacrylate](POEGMA-Hyd)

The carboxylic acid functional groups of POEGMA-AA were subsequently converted to hydrazide groups via a carbodiimide-mediated conjugation of a large excess of adipic acid dihydrazide (ADH). The polymer (4.0 g) was dissolved in 100 mL of DIW and added to a 250 mL round-bottom flask. ADH (7.32 g, 41.8 mmol, 5 mol eq.) was added, and the pH of the solution was adjusted to pH=4.75 using 0.1 M HCl. Subsequently, EDC (3.27 g, 20.9 mmol, 2.5 mol eq.) was added, and the pH was maintained at pH=4.75 by the dropwise addition of 1.0 M HCl over 4 hours. The solution was left to stir overnight, dialyzed against DIW for a minimum of 6 (6+ hour) cycles, and lyophilized. The degree of functionalization was determined using conductometric base-into-acid titration. The polymers were stored as 20 w/w % solutions in DIW with pH 7.4 at 4° C.

### Synthesis of Poly[Oligo(Ethylene Glycol) Methyl Ether Methacrylate]-Hydrazine-Dimethylmaleic Acid Copolymers (POEGMA-Hyd-DMA)

POEGMA-Hyd was further reacted with 2,3-dimethylmaleic anhydride via a nucleophilic addition-elimination reaction. The polymer (4.0 g) was dissolved in 50 mL of DIW and the pH of the solution was adjusted to pH 10.0 using 1.0 M NaOH and added to a 250 mL round-bottom flask. DMA (2.2 g, 17.4 mmol, 2 mol eq.) was added, and the pH was maintained at 9.0 by the dropwise addition of 1.0 M NaOH over 4 hours. The solution was left to stir overnight, dialyzed against DIW at pH of 9.0 for a minimum of 6 (6+ hour) cycles, and lyophilized. The polymers were stored as 20 w/w % solutions in DIW with pH 7.4 at 4° C.

Synthesis of Aldehyde-Functionalized Cationic Starch Nanoparticles (cSNP-CHOs)

Cationic SNPs (cSNPs, 2.0 g) were dissolved in 60 mL of DIW/DMF (1:2), and the pH of the solution was adjusted to pH 9.0 using 1.0 M NaOH. Bromobenzaldehyde (8.0 g) was then added, and the pH was maintained at pH=9.0 by the dropwise addition of 1.0 M NaOH over 4 hours. The solution was left to stir overnight, dialyzed against DIW for a minimum of 6 (6+ hour) cycles, and lyophilized.

### Characterization

### Gel Permeation Chromatography (GPC)

Gel permeation chromatography (GPC) was carried out using Phenomenex Phenogel columns (300 mm×4.6 mm, 5 m; pore size 100, 500, 10⁴ A) at room temperature; DMF with 50 mM LiBr was used as the eluent at a flow rate of 0.3 mL/min and calibrated using PEG narrow standards obtained from Polymer Laboratories. All samples were filtered using a 0.2 m nylon filter. ¹H-NMR was performed on a Bruker AVANCE 600 MHz spectrometer using deuterated dimethyl sulfoxide as the solvent.

### Quantification of Acrylic Acid Content

The acrylic acid content of the polymers was determined using base-into-acid conductometric titration (ManTech Associates) using 50 mg of polymer dissolved in 50 mL of 1 mM NaCl as the analysis sample and 0.1 M NaOH as the titrant.

Size Distribution of cSNPs and cSNP-CHOs with Dynamic Light Scattering (DLS)

cSNP-CHOs were dispersed in 5 mM NaCl solution and measured on a NanoBrook 90Plus dynamic light scattering instrument (Brookhaven, Long Island, N.Y., USA; temperature=37° C.).

### Zeta Potential and Electrophoretic Mobility

cSNP-CHOs were dispersed in 5 mM NaCl solution, and their electrophoretic mobility was measured on a NanoBrook 90Plus instrument operating in phase analysis light scattering (PALS) mode (Brookhaven, Long Island, N.Y., USA; temperature=37° C.).

### Results and Discussion

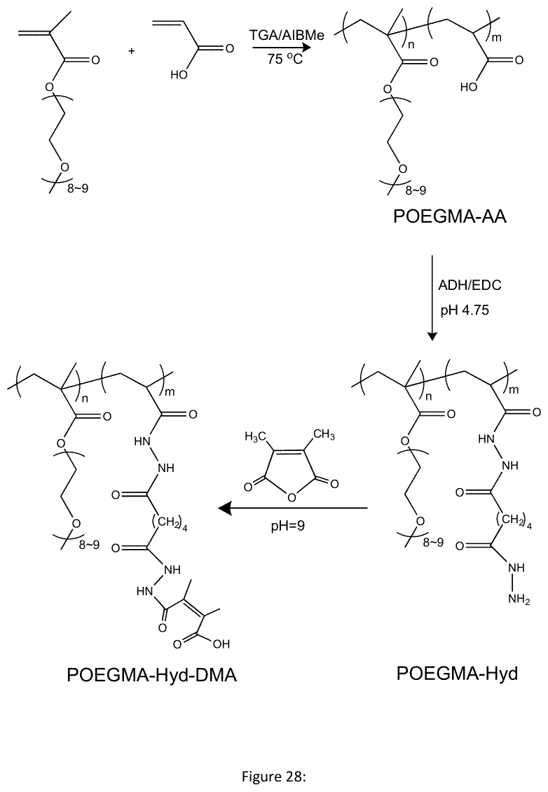

POEGMA-AA was synthesized by conventional free radical polymerization of OEGMA and AA with a chain transfer agent to limit the molecular weight to below the renal cut-off (number average molecular weight of 12-30 kDa). POEGMA-Hyd was synthesized by conjugation of ADH using carbodiimide-catalyzed coupling to POEGMA-AA. POEGMA-Hyd-DMA was synthesized by the nucleophilic addition-elimination reaction between the hydrazide groups and DMA. The syntheses performed and the chemical structures of the POEGMA copolymers were depicted in FIG. 28.

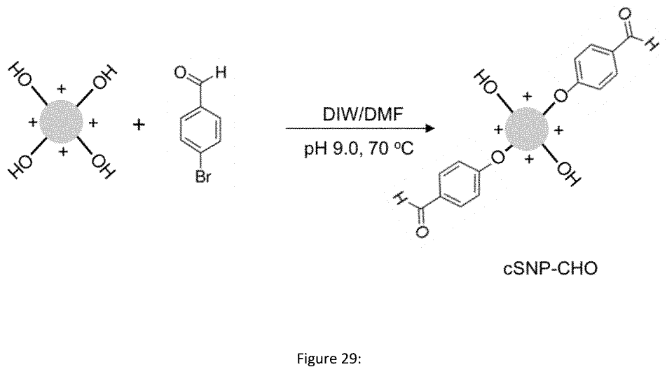

cSNP-CHOs were synthesized by a substitution reaction to conjugate bromobenzaldehyde to the hydroxyl groups on the starch nanoparticles. The synthesis performed and the chemical structure of cSNP-CHOs were depicted in FIG. 29.

The hydrodynamic diameter of cSNP-CHOs was 10-50 nm, slightly smaller than the particle size range of SNP-CHOs. The surface zeta potential of cSNP-CHOs was +5 to +40 mV, confirming the maintained cationic charge.

### Example C2: Synthesis and Physiochemical Characterization of Smart Nanoclusters Doxorubicin-Loaded Aldehyde-Functionalized Cationic Starch Nanoparticles (DOX-cSNPs)

cSNP-CHOs (1.0 g) were suspended in 10 mL of DIW, and doxorubicin (100 mg) was dissolved in 1 mL of DMSO. DOX solution was added into cSNP suspension, and the solution was left to stir overnight in the dark, dialyzed against DIW for a minimum of 6 (6+ hour) cycles, and lyophilized.

### Preparation of Doxorubicin-Loaded Smart Nanoclusters (DOX-iCluster)

Doxorubicin-loaded smart nanocluster nanogels were prepared by mixing the positively charged DOX-cSNPs and negatively charged pH-responsive POEGMA-Hyd-DMA (smart nanocluster nanogels, DOX-iClusters) or non-pH-responsive POEGMA-AA (control nanocluster nanogels, DOX-Clusters). All precursors were initially dissolved in water (pH=10.0) and magnetically mixed at 1500 rpm for 4 h to create the ionically-crosslinked nanocluster nanogels. The DOX-iCluster solutions for in vitro tests were prepared by dialyzing the mixture against DIW (pH=7.4) for seven days prior to testing to remove any unencapsulated drug.

### Characterization

Size Distribution with Dynamic Light Scattering (DLS)

Nanocluster nanogels were dispersed in 5 mM NaCl solution, and the particle size was measured using a NanoBrook 90Plus dynamic light scattering instrument (Brookhaven, Long Island, N.Y., USA; temperature=37° C.).

### Transmission Electron Microscopy (TEM)

Transmission electron microscopy (TEM) was performed using a JEOL 1200EX TEMSCAM instrument to access nanocluster nanogel size and structure. Nanocluster nanogels were deposited and dried on a carbon coated copper grid.

### Doxorubicin (DOX) Loading Test

The ultraviolet-visible (UV-vis) absorbance spectra of DOX-loaded nanoclusters solutions were quantitatively measured using a Beckman Coulter DU 800 spectrophotometer operating in a wavelength range of 200-900 nm. An absorbance wavelength of 490 nm was used to record the absorbance, with the DOX concentration calculated based on a calibration curve.

### Results and Discussion

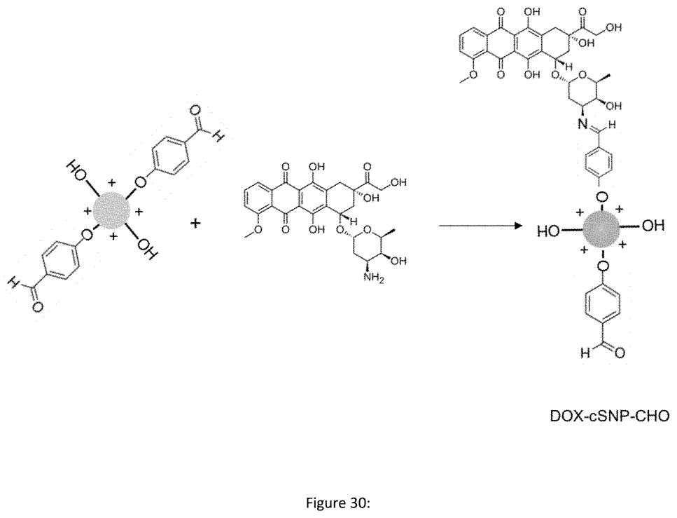

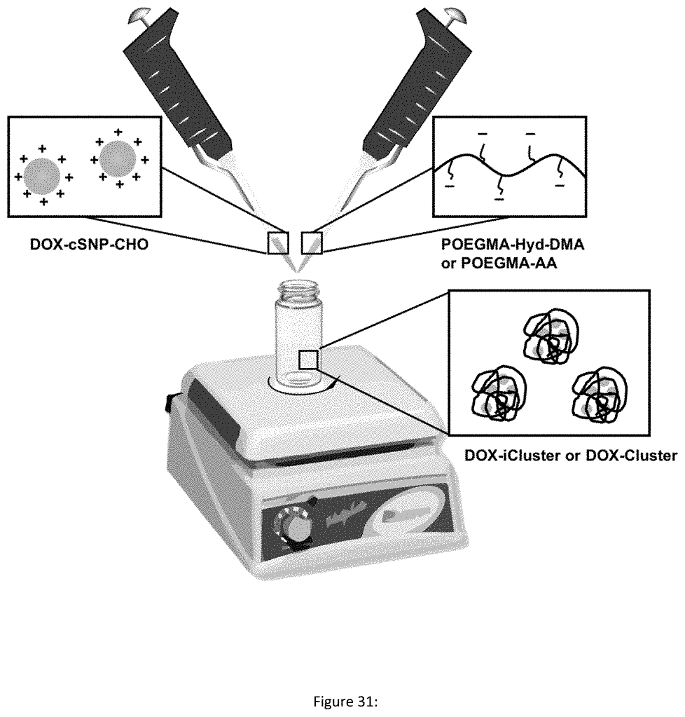

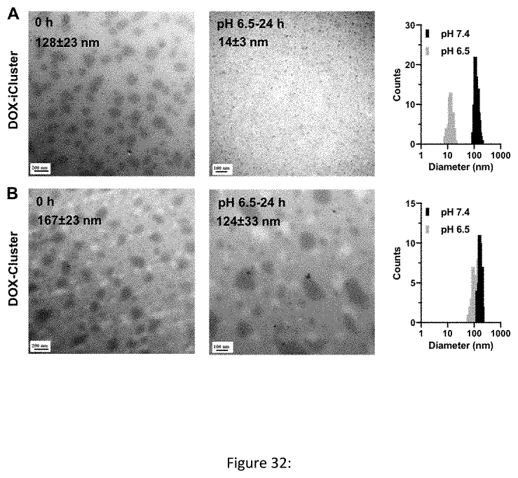

Doxorubicin-loaded smart nanoclusters were prepared by mixing 1, 100, or 300 mg/mL of DOX-cSNP-CHOs (FIG. 30) and POEGMA-Hyd-DMA (1:9 w/w ratio to DOX-cSNP-CHOs) in distilled deionized water (FIG. 31). The hydrodynamic diameter of the DOX-iCluster at pH 7.4 was 128±23 nm (70-250 nm particle size range), while the hydrodynamic diameter of the control DOX-Cluster at pH 7.4 was 167±23 nm (70-250 nm range) (FIG. 32). After changing the pH to 6.4 for 24 hrs to simulate the tumoral microenvironment, the hydrodynamic diameter of the charge-switching DOX-iCluster decreased to 14±3 nm (10-50 nm range), consistent with that of free cSNPs; in contrast, the hydrodynamic diameter of the fixed charge DOX-Cluster remained at 133±33 nm (70-250 nm range). This result suggests that the smart charge-reversible iClusters can degrade at moderately acidic pH values consistent with the tumor microenvironment due to their capacity for charge switching, as the control cluster does not substantially degrade under the same conditions.

### Example C3: Biological Applications of Smart Nanocluster Designs

### In Vitro Cell Viability Assay:

The cytotoxicity of the fabricated nanoclusters towards B16-F10 melanoma cells with or without DOX loading was assayed in 96 well plates. 200 μL of the cell suspension (2.5×10⁴ cells/mL) in DMEM medium was seeded into the wells of a 96 well plate. One day after the cell attachment, free DOX (control), DOX-iCluster and DOX-Cluster suspensions of various DOX concentrations (0, 0.01, 0.05, 0.1, 0.5, 1.0 and 5.0 μg/mL) were added into separate wells. After 24 hrs and 72 hrs of incubation, the media was removed and resazurin solutions pre-dissolved in media were added into the wells as per the manufacturer's instructions. After 4 h of incubation, the fluorescence intensity was measured using a Tecan Infinite M200 Pro plate reader using an excitation wavelength of 560 nm and an emission wavelength of 590 nm. All measurements were done in triplicate, with the error bars representing the standard deviation of the replicate measurements.

### In Vitro Cells Spheroid Penetration Assay:

The penetration capacity of nanoclusters was tested using B16-F10 melanoma cell spheroids. 10 μL of the cell suspension (10⁵ cells/mL) was dropped on the lid of the petri dish and left for 7 days to promote spheroid formation using the hanging drop method. 1 μL of DOX-iCluster or DOX-Cluster suspensions was added into the cell spheroids at either pH 7.4 (normal physiological conditions) and pH 6.5 (tumoral pH conditions). After 3 hrs of incubation, the media was removed and the spheroids were washed with PBS to removed free (non-adhered) nanoclusters. The spheroids were assayed using confocal laser scanning microscopy (CLSM, Nikon) using an excitation wavelength of 488 nm, an emission wavelength of 530 nm, and a bandwidth of 20 nm to directly image and compare the DOX-related fluorescence intensity of the spheroids at different depths.

### In Vivo Anti-Tumor Test:

The antitumor effect of DOX, DOX-Clusters (control), and Dox-iClusters was evaluated by tracking the relative tumor volume (V/V₀) after treatment with the nanocluster nanogels. C57BL/6 mice were injected intradermally with B16-F10 melanoma tumor cells, allowing the tumors to grow to ˜20 mm³ by day 7 after the injections. The mice were treated by tail vein injection of 200 μL of PBS, DOX, DOX-iCluster and DOX-Cluster every three days (5.0 mg/kg of DOX for all formulations), after which both the tumor size and body weight were monitored regularly over a 10-day period.

### Results and Discussion

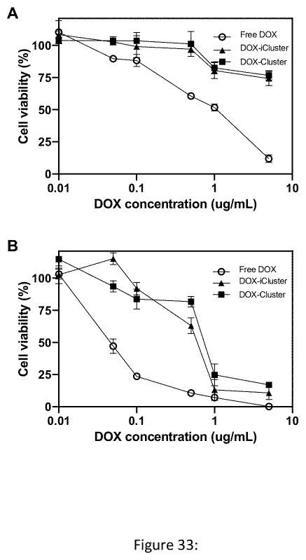

The viability of B16-F10 melanoma cells after incubation with DOX-iClusters and DOX-Clusters for 24 hr was higher than that of the free DOX (FIG. 33-A), consistent with nanocluster encapsulation leading to slower DOX release. A significant decrease in cell viability was noted after 72 hrs (FIG. 33-B) in the DOX-iCluster and DOX-Cluster groups consistent with sufficient drug being released over time to effectively kill the tumor cells.

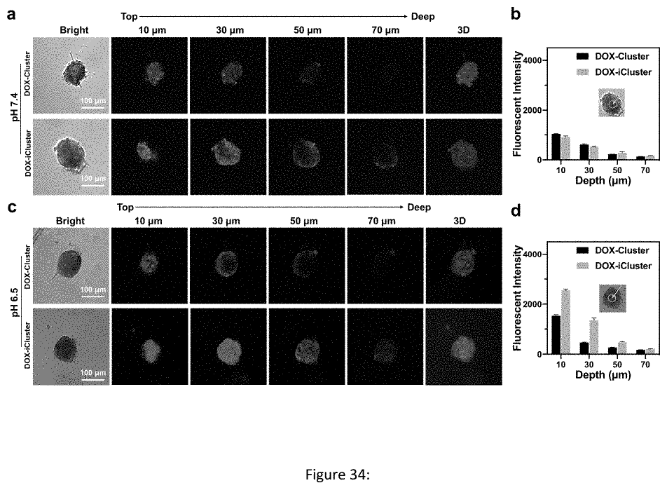

DOX penetration into the B16-F10 spheroid with DOX-iCluster and DOX-Cluster was minimal at pH 7.4 (FIG. 34-A,B), suggesting these formulations were stable with minimal DOX release at physiological pH. However, a significant increase in spheroid DOX penetration was noted in the DOX-iCluster at pH 6.5 (FIG. 34-C, D), while the DOX penetration from the control DOX-Cluster did not significantly change relative to that observed at physiological pH. This result is consistent with the smart DOX-iClusters degrading to release the small and highly penetrative cSNPs at tumoral pH and thus increase the drug penetration into the tumor.

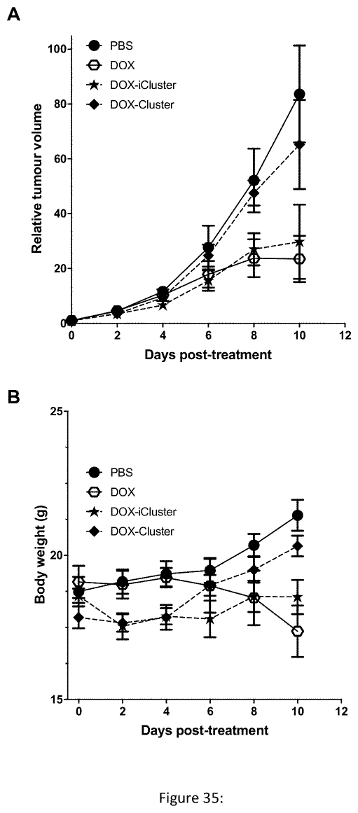

The anti-tumor effects of free DOX, DOX-loaded smart nanocluster (DOX-iCluster) and DOX-loaded control nanocluster (DOX-Cluster) were evaluated by measuring the relative tumor volume over time following intradermal implantation of B16-F10 tumor cells (FIG. 35-A). Compared with the PBS group (84× volume increase), there was a significantly reduced relative tumor volume in the DOX-iCluster group (29× volume increase) that was comparable that observed in the free DOX group (24× volume increase). However, the free DOX group also showed significantly reduced body weight over the ten days of tracking due to the toxicity of this concentration of free DOX to normal organs (FIG. 35-B), a weight reduction that was not observed with the DOX-iCluster group. This result indicate that the DOX-iCluster had significantly lower adverse effects from chemotoxicity on the normal organs without sacrificing tumor killing ability. In comparison, there was no significant reduction in the relative tumor volume for the DOX-Cluster group (65.2×) relative to the PBS control, suggesting the triggered cSNP release enabled by the pH-switchable DOX-iCluster can lead to improved anti-tumor effect.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term

TABLE 1
Gelation time of bulk gels with varying compositions of
CS-SH, unfunctionalized SNPs, SNP-CHO, SNP-SH, and GSSG.

| | | | | Gelation |
|---|---|---|---|---|
| | [CS-SH] | [SNP derivative] | [GSSG] | Time |
| | (w/v %) | (w/v %) | (mM) | (mins) |
| CS-SH@CS-SH | 10 | — | — | 260 |
| | 20 | — | — | 300 |
| | 30 | — | — | 240 |
| | 15 | — | 1 | 75 |
| | 15 | — | 5 | 25 |
| | 15 | — | 10 | 9 |
| CS-SH@SNP-SH | 5 | 5 | — | 1440 |
| | 10 | 10 | — | 1440 |
| | 15 | 15 | — | 1440 |
| | 10 | 1 | 5 | 180 |
| | 15 | 1 | 5 | 75 |
| | 20 | 1 | 5 | 42 |
| | 15 | 1 | 5 | 60 |
| | 15 | 5 | 5 | 240 |
| | 15 | 10 | 5 | 300 |
| SNP-SH@SNP-SH | — | 10 | — | — |
| | — | 20 | — | — |
| | — | 30 | — | — |
| CS-SH@SNP | 10 | 1 | 5 | 165 |
| | 15 | 1 | 5 | 75 |
| | 20 | 1 | 5 | 25 |
| | 15 | 1 | 5 | 75 |
| | 15 | 5 | 5 | 3 |
| | 15 | 10 | 5 | 1 |
| CS-SH@SNP-CHO | 10 | 1 | 5 | 95 |
| | 15 | 1 | 5 | 75 |
| | 20 | 1 | 5 | 25 |
| | 15 | 1 | 5 | 10 |
| | 15 | 5 | 5 | 1.5 |
| | 15 | 10 | 5 | <1 |

### REFERENCES

1. Majcher, M. J.; Hoare, T. “Advanced Hydrogel Structures” in Springer Functional Biopolymers. Springer, 2019, DOI:10.1007/978-3-319-92066-5_16-1.
2. El.-Sherbiny, I. M.; Yacoub, M. H. Hydrogel Scaffolds for Tissue Engineering: Progress and Challenges. Glob. Cardiol. Sci. Pract. 2013, 2013 (3). 316-342.
3. Peppas, N. A.; Snelling van Blarcom, D. Hydrogel-Based Biosensors and Sensing Devices for Drug Delivery. J Control. Release 2016, 240. 142-150.
4. Hamidi, M.; Azadi, A.; Rafiei, P. Hydrogel Nanoparticles in Drug Delivery. Adv. Drug Deliv. Rev. 2008, 60 (15). 1638-1649.
5. Campea, M. A.; Majcher, M. J.; Lofts, A.; Hoare, T. A Review of Design and Fabrication Methods for Nanoparticle Network Hydrogels (NNHs) for Biomedical, Environmental, and Industrial Applications. Adv. Funct. Mater. 2021. In Press.
6. Bo, J.; Luo, X.; Huang, H.; Li, L.; Lai, W.; Yu, X. Morphology-Controlled Fabrication of Polypyrrole Hydrogel for Solid-State Supercapacitor. J. Power Sources 2018, 407, 105-111.
7. Su, X.; Chen, B. Tough, Resilient and pH-Sensitive Interpenetrating Polyacrylamide/Alginate/Montmorillonite Nanocomposite Hydrogels. Carbohydr. Polym. 2018, 197, 497-507.
8. Rashidzadeh, A.; Olad, A.; Salari, D.; Reyhanitabar, A. On the Preparation and Swelling Properties of Hydrogel Nanocomposite Based on Sodium Alginate-g-Poly (Acrylic Acid-Co-Acrylamide)/Clinoptilolite and Its Application as Slow Release Fertilizer. J. Polym. Res. 2014, 21 (2), 344.
9. Rashidzadeh, A.; Olad, A.; Reyhanitabar, A. Hydrogel/Clinoptilolite Nanocomposite-Coated Fertilizer: Swelling, Water-Retention and Slow-Release Fertilizer Properties. Polym. Bull. 2015, 72 (10), 2667-2684.
10. Yang, Y.; Zhao, Y.; Sun, S.; Zhang, X.; Duan, L.; Ge, X.; L0, W. Self-Assembled Three-Dimensional Graphene/Fe₃O₄ Hydrogel for Efficient Pollutant Adsorption and Electromagnetic Wave Absorption. Mater. Res. Bull. 2016, 73, 401-408.
11. Wang, J.; Lin, L.; Cheng, Q.; Jiang, L. A Strong Bio-Inspired Layered PNIPAM-Clay Nanocomposite Hydrogel. Angew. Chem. Int. Ed. 2012, 51 (19), 4676-4680.
12. Motamedi, E.; Motesharezedeh, B.; Shirinfekr, A.; Samar, S. M. Synthesis and Swelling Behavior of Environmentally Friendly Starch-Based Superabsorbent Hydrogels Reinforced with Natural Char Nano/Micro Particles. J. Environ. Chem. Eng. 2020, 8 (1), 103583.
13. Perrault, S. D.; Walkey, C.; Jennings, T.; Fischer, H. C.; Chan, W. C. W. Mediating Tumor Targeting Efficiency of Nanoparticles Through Design. Nano Lett. 2009, 9 (5), 1909-1915.
14. Wang, J.; Mao, W.; Lock, L. L.; Tang, J.; Sui, M.; Sun, W.; Cui, H.; Xu, D.; Shen, Y. The Role of Micelle Size in Tumor Accumulation, Penetration, and Treatment. ACS Nano 2015, 9 (7), 7195-7206.
15. Albanese, A.; Tang, P. S.; Chan, W. C. W. The Effect of Nanoparticle Size, Shape, and Surface Chemistry on Biological Systems. Annu. Rev. Biomed. Eng. 2012, 14 (1), 1-16.
16. Perrault, S. D.; Walkey, C.; Jennings, T.; Fischer, H. C.; Chan, W. C. W. Mediating Tumor Targeting Efficiency of Nanoparticles Through Design. Nano Lett. 2009, 9 (5), 1909-1915.
17. Wong, C.; Stylianopoulos, T.; Cui, J.; Martin, J.; Chauhan, V. P.; Jiang, W.; Popovic, Z.; Jain, R. K.; Bawendi, M. G.; Fukumura, D. Multistage Nanoparticle Delivery System for Deep Penetration into Tumor Tissue. Proc. Natl. Acad. Sci. U.S.A. 2011, 108 (6), 2426-2431.
18. Gulzar, A.; Xu, J.; Wang, C.; He, F.; Yang, D.; Gai, S.; Yang, P.; Lin, J.; Jin, D.; Xing, B., Tumour microenvironment responsive nanoconstructs for cancer theranostic. Nano Today 2019. 26.16-56.
19. Li, H.-J.; Du, J.-Z.; Liu, J.; Du, X.-J.; Shen, S.; Zhu, Y.-H.; Wang, X.; Ye, X.; Nie, S.; Wang, J., Smart superstructures with ultrahigh pH-sensitivity for targeting acidic tumor microenvironment: instantaneous size switching and improved tumor penetration. ACS Nano 2016, 10 (7), 6753-6761.
20. Kim, J.; Jo, C.; Lim, W. G.; Jung, S.; Lee, Y. M.; Lim, J.; Lee, H.; Lee, J.; Kim, W. J., Programmed nanoparticle-loaded nanoparticles for deep-penetrating 3D cancer therapy. Adv Mater 2018, 30 (29), 1707557.
21. Lo, Y.-L.; Sung, K.-H.; Chiu, C.-C.; Wang, L.-F. Chemically Conjugating Polyethylenimine with Chondroitin Sulfate to Promote CD44-Mediated Endocytosis for Gene Delivery. Mol. Pharm. 2013, 10 (2), 664-676.
22. Adekola, K.; Rosen, S. T.; Shanmugam, M. Glucose Transporters in Cancer Metabolism. Curr. Opin. Oncol. 2012, 24 (6), 650-654.
23. Chen, B.; Le, W.; Wang, Y.; Li, Z.; Wang, D.; Lin, L.; Cui, S.; Hu, J. J.; Hu, Y.; Yang, P.; Ewing, R. C.; Shi, D.; Cui, Z. Targeting Negative Surface Charges of Cancer Cells by Multifunctional Nanoprobes. Theranostics 2016, 6 (11), 1887-1898.
24. Blair, S. L.; Heerdt, P.; Sachar, S.; Abolhoda, A.; Hochwald, S.; Cheng, H.; Burt, M. Glutathione Metabolism in Patients with Non-Small Cell Lung Cancers. Cancer Res. 1997, 57 (1), 152-155.
25. Gamcsik, M. P.; Kasibhatla, M. S.; Teeter, S. D.; Colvin, O. M. Glutathione Levels in Human Tumors. Biomarkers 2012, 17 (8), 671-691.
26. Yan, Y.; Zuo, X.; Wei, D. Concise Review: Emerging Role of CD44 in Cancer Stem Cells: A Promising Biomarker and Therapeutic Target. Stem Cells Transl. Med. 2015, 4 (9), 1033-1043.
27. Vene, R.; Castellani, P.; Delfino, L.; Lucibello, M.; Ciriolo, M. R.; Rubartelli, A. The Cystine/Cysteine Cycle and GSH Are Independent and Crucial Antioxidant Systems in Malignant Melanoma Cells and Represent Druggable Targets. Antioxid. Redox Signal. 2011, 15 (9), 2439-2453.
28. Wu, W.; Luo, L.; Wang, Y.; Wu, Q.; Dai, H.-B.; Li, J.-S.; Durkan, C.; Wang, N.; Wang, G.-X. Endogenous PH-Responsive Nanoparticles With Programmable Size Changes for Targeted Tumor Therapy and Imaging Applications. Theranostics 2018, 8 (11). 3038-3058
29. Cole, A. S.; Eastoe, J. E.; Cole, A. S.; Eastoe, J. E. Carbohydrates. Biochem. Oral Biol. 1988, 90-99.
30. Slavin, J.; Carlson, J. Carbohydrates. Adv. Nutr. 2014, 5 (6), 760-761.
31. Sajilata, M. G.; Singhal, R. S.; Kulkarni, P. R. Resistant Starch? A Review. Compr. Rev. FoodSci. Food Saf 2006, 5 (1), 1-17.
32. Ismail, H.; Irani, M.; Ahmad, Z. Starch-Based Hydrogels: Present Status and Applications. Int. J. Polym. Mater. 2013, 62 (7), 411-420.
33. Chrungoo, N. K.; Devi, A. G.; Chrungoo, N. K.; Devi, A. G. Morphological and Rheological Properties of Starches Separated from Cultivars of Rice from North East India. Am. J. Plant Sci. 2015, 6 (12), 2019-2031.
34. Ellis, R. P.; Cochrane, M. P.; Dale, M. F. B.; Duffus, C. M.; Lynn, A.; Morrison, I. M.; Prentice, R. D. M.; Swanston, J. S.; Tiller, S. A. Starch Production and Industrial Use. J. Sci. Food Agric. 1998, 77 (3), 289-311.
35. Baran, E. T.; Mano, J. F.; Reis, R. L. Starch-Chitosan Hydrogels Prepared by Reductive Alkylation Cross-Linking. J. Mater. Sci. Mater. Med. 2004, 15 (7). 759-765.
36. Marques, A. P.; Reis, R. L.; Hunt, J. A. The Biocompatibility of Novel Starch-Based Polymers and Composites: In Vitro Studies. Biomaterials 2002, 23 (6), 1471-1478.
37. Na, J. H.; Lee, S.-Y.; Lee, S.; Koo, H.; Min, K. H.; Jeong, S. Y.; Yuk, S. H.; Kim, K.; Kwon, I. C. Effect of the Stability and Deformability of Self-Assembled Glycol Chitosan Nanoparticles on Tumor-Targeting Efficiency. J. Control. Release 2012, 163 (1), 2-9.
38. Latreille, P.-L.; Adibnia, V.; Nour, A.; Rabanel, J.-M.; Lalloz, A.; Arlt, J.; Poon, W. C. K.; Hildgen, P.; Martinez, V. A.; Banquy, X. Spontaneous Shrinking of Soft Nanoparticles Boosts Their Diffusion in Confined Media. Nat. Commun. 2019, 10 (1), 4294.
39. Majima, Y.; Harada, T.; Shimizu, T.; Takeuchi, K.; Sakakura, Y.; Yasuoka, S.; Yoshinaga, S. Effect of Biochemical Components on Rheologic Properties of Nasal Mucus in Chronic Sinusitis. Am. J. Respir. Crit. Care Med. 1999, 160 (2). 421-426
40. Liu, Y.; Johnson, M. R.; Matida, E. A.; Kherani, S.; Marsan, J. Creation of a Standardized Geometry of the Human Nasal Cavity. J. Appl. Physiol. 2009, 106 (3), 784-795.
41. Sun, Z. Starch Nanoparticles: Modifications, Toxicity, and Drug Loading. Master's Thesis, University of Waterloo, 2015.

1. A hydrogel composition, comprising
a. at least one polysaccharide-based nanoparticle functionalized with one or more first functional moieties; and
b. at least one polymer functionalized with one or more second functional moieties,
wherein at least one of the first functional moieties and at least one of the second functional moieties are crosslinked through covalent and/or physical crosslinks to form the hydrogel composition.

2. The hydrogel composition of claim 1, wherein each dimension of the hydrogel is greater than about 1 mm.

3. The hydrogel composition of claim 1, wherein the hydrogel is a microparticle with at least one dimension less than one millimetre, a nanoparticle with at least one dimension less than one micrometre, or another kind of particulate form.

4. The hydrogel composition of claim 1, wherein the sizes of the polysaccharide-based nanoparticle and the hydrogel particle are selected to enable different biological responses.

5. The hydrogel composition of claim 4, wherein the polysaccharide nanoparticle is less than about 50 nm in size and the hydrogel particle is between about 50 nm to about 1000 nm in size.

6. The hydrogel composition of claim 1, wherein the first functional moiety is a nucleophilic moiety and the second functional moiety is an electrophilic moiety.

7. The hydrogel composition of claim 1, wherein the first functional moiety is an electrophilic moiety and the second functional moiety is a nucleophilic moiety.

8. The hydrogel composition of claim 1, wherein the covalent crosslink is a Schiff base bond.

9. The hydrogel composition of claim 1, wherein the covalent crosslink is a disulfide bond.

10. The hydrogel composition of claim 1, wherein the physical crosslink is an ionic interaction.

11. The hydrogel composition of claim 1, wherein the first functional moiety is an aldehyde or derivative thereof, a sulfide, a carboxylic acid, an amino group, a phenylboronic acid, a cationic group, an anionic group, and/or a hydrophobic moiety.

12. The hydrogel composition of claim 1, wherein the aldehyde moiety is a bromobenzaldehyde moiety.

13. The hydrogel composition of claim 1, wherein the polysaccharide-based nanoparticle is a starch-based nanoparticle.

14. The hydrogel composition of claim 1, wherein the second functional moiety is an aldehyde or derivative thereof, a sulfide, a carboxylic acid, an amino group, phenylboronic acid, a cationic group, an anionic group, and/or a hydrophobic moiety.

15. The hydrogel composition of claim 1, comprising
a. a starch-based nanoparticle functionalized with aldehyde groups; and
b. chitosan, carboxymethyl chitosan, or a derivative thereof,
wherein the hydrogel is formed from reversible imine bonds.

16. The hydrogel composition of claim 1, comprising
a. a starch-based nanoparticle functionalized with thiol and/or aldehyde groups;
b. chondroitin sulfate functionalized with thiol groups,
wherein the hydrogel is formed from reversible disulfide or thioacetal bonds.

17. The hydrogel composition of claim 1, comprising
a. a cationic starch-based nanoparticle;
b. a poly[oligo(ethylene glycol) methyl ether methacrylate] functionalized with carboxylic acid groups,
wherein the hydrogel is formed from cationic-anionic interactions.

18. The hydrogel composition of claim 13 in which the cationic starch-based nanoparticle is also aldehyde-functionalized.

19. The hydrogel composition of claim 1, wherein the crosslinking is reversible over time and/or in response to one or more environmental stimuli, including but not limited to pH, temperature, ionic strength, or the concentration of a particular chemical.

20. The hydrogel composition of claim 1, wherein the polymer is a thiolated glycosaminoglycan polymer.

21. The hydrogel composition of claim 16, wherein the thiolated glycosaminoglycan polymer is chondroitin sulfate A, chondroitin sulfate B, chondroitin sulfate C, hyaluronic acid, heparan sulfate, heparin, keratan sulfate, and their salts and their derivatives.

22. A method for the administration of a hydrogel composition of claim 1 containing a therapeutic agent for the treatment of a condition, in which the polysaccharide-based nanoparticle and the crosslinking polymer are co-administering to a patient to enable the in situ formation of the hydrogel composition.

23. The method of claim 18, wherein the precursor components of the hydrogel are administered via the intravenous, intramuscular, intracranial, subcutaneous, intradermal, or intranasal routes.

24. The method of claim 1 wherein the hydrogel composition is used to physically encapsulate and/or chemically bond the therapeutic agent to treat a condition.
